(19) United States
(12) Patent Application Publication
(10) Pub. No.: US 2014/0322741 A1
(43) Pub. Date: Oct. 30, 2014

(54) Cell Free CD4 Quantitation and Methods of Use

(71) Applicant: Alpha 1 Biologics Corporation, Stony Brook, NY (US)

(72) Inventor: Cynthia L. Bristow, Long Beach, NY (US)

(21) Appl. No.: 14/326,163

(22) Filed: Jul. 8, 2014

Related U.S. Application Data
(63) Continuation of application No. 13/450,532, filed on Apr. 19, 2012.

(51) Int. Cl. C12Q 1/37

(52) U.S. Cl. CPC C12Q 1/37
USPC 435/23

(57) ABSTRACT

The present invention provides a low-cost cell-free assay, the α-test, that provides point-of-care CD4 enumeration using a single platform assay thereby eliminating the need for high-end instrumentation, calibrated pipetting, and specialized technical training. The number of CD4 T cells in blood is driven by the concentration of the protein α1 proteinase inhibitor (α1PI, α1 antitrypsin, serpin A1). The invention features, in part, methods for determining the number of CD4+ T cells in a sample comprising determining the concentration of alpha 1 proteinase inhibitor (α1PI) in a sample; wherein the number of CD4+ T-cells is related to the concentration of α1PI.

### CROSS-RELATED APPLICATIONS/PATENTS & INCORPORATION BY REFERENCE

This application is a continuation of International Patent Application No.: PCT/US2010/053717, filed on Oct. 22, 2012, which claims the benefit of the U.S. Provisional Application No. 61/279,538, filed Oct. 22, 2009, the disclosure of each which is incorporated herein by reference in its entirety for all purposes.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these application and patents, and each of the documents cited or referenced in each of the application cited documents, are hereby expressly incorporated herein by reference. More generally, documents or references are cited in this text, either in a Reference List before the claims, or in the text itself; and, each of these documents or references (“herein-cited references”), as well as each document or reference cited in each at the herein-cited references (including any manufacturer's specifications, instructions, etc.), is hereby expressly incorporated herein by reference.

### BACKGROUND OF THE INVENTION

Stem cell migration from Drosophila to humans requires LDL receptor-mediated Wnt-induced signaling (Cselenyi et al., 2008), and in humans requires α1 proteinase inhibitor (α1PI, α1antitrypsin, serpin A1) (Goselink et al., 1996), its receptor cell surface human leukocyte elastase (HLECS) (Bristow et al., 2003; Lapidot and Petit, 2002), the chemokine CXCL12 (SDF-1), and its receptor CXCR4 (Lapidot and Petit, 2002), the same components involved in HIV-1 uptake (Bristow et al., 2003). It has long been know that coupling of active α1PI to soluble HLE inactivates both proteins and exposes the C-terminal domain of α1PI (C-36, VIRIP) which then binds to receptors for low density lipoprotein (LDL) (Cao et al., 2006). Yet, it has not been appreciated that HLE is also localized on the cell surface (Bristow et al., 1995), and that when α1PI binds to HLECS at the leading edge of a migrating cell, the complex induces receptor polarization (Bristow et al., 2003). Due to forward movement of the cell, receptor complexes underneath the cell reposition in “millipede-like locomotion” (Shulman et al., 2009) to the trailing edge where α1PI binds to LDL receptors, a condition that induces endocytosis of the complex and retraction of the trailing edge, and recycling of receptors to the leading edge of the migrating cell via endosomes in conveyor belt type motion (Cao et al., 2006).

If one of the components involved in this conveyor belt mechanism is missing or blocked, the cell halts migrating. For example, bacteria, snake bites, blood clotting, and most other non-normal situations produce non-normal proteases which cleave sentinel proteinase inhibitors including α1PI which is the most abundant proteinase inhibitor in serum. When α1PI is inactivated, it can no longer bind its receptor HLECS. In the absence of α1PI-HLECS complexes, the LDL receptors are not triggered for endocytosis and this causes blood cells to stop migrating. CD4+ lymphocytes, the helper immune cells, are the third most abundant cells in blood after red blood cells and neutrophils. Thus, a cut on the skin, a low-grade bacterial infection in an artery, or other pathologic situations cause CD4+ lymphocytes to stop migrating and clump up exactly at the site of the pathology where “help” is needed. On the contrary, a leukemic cell or a virus-containing cell would not interrupt cell migration or initiate an immune response unless the state of homeostasis is tipped by cell lysis, redox, or other abnormal changes.

Human immunodeficiency virus type 1 (HIV-1) infects cells that express the CD4 receptor and depletes its host of CD4 lymphocytes. This depletion of CD4 T lymphocytes has been linked to the immunopathogenesis of HIV infection and progression of the disease. CD4 T-lymphocyte measurements have been used to predict the onset of opportunistic diseases, such as Pneumocystis carinii pneumonia, and to monitor immune reconstitution.

The current method for determining absolute CD4 T-lymphocyte counts is dependent upon immunophenotypic identification of cells with fluorescently labelled monoclonal antibodies directed against the CD4 antigen, where relative percentages of CD4 T cells are determined with a flow cytometer. An absolute CD4 count is derived by multiplying the percentage of lymphocytes that are CD3+ CD4+ by the absolute lymphocyte count determined with a hematology instrument. Error arises in the method both in the shipment of samples and in the variability of the instrumentation that is used.

Accordingly, there is a need better CD4 enumeration, both domestically as well as in resource-limited regions. Further, a low cost assay that provides point-of-care enumeration and eliminates the need for high-end instrumentation and specialized technical training would benefit many populations, including those that are presently unable to afford or logistically obtain care.

### SUMMARY OF THE INVENTION

In the present invention a low-cost cell-free assay, the α-test, is described that provides point-of-care enumeration of CD4+ T lymphocytes in the blood of HIV patients. New evidence shows that the number of CD4+ T lymphocytes in blood is driven by the concentration of the protein α1 proteinase inhibitor (α1PI, α1 antitrypsin, serpin A1). Accordingly, in the present invention, the linear relationship between α1PI and CD4+ lymphocytes, measured in serum or saliva, allows the use of a mathematical formula to produce accurate CD4 T cell counts.

The present invention also describes that preconditioning cells with active α1PI for 15 min is required for receptor polarization and HIV-1 infectivity, but preconditioning for 60 min exhausts polarization and inhibits HIV-1 infectivity. Blocking very low density lipoprotein receptor (VLDLR) blocks internalization CD4-complexed HIV-1 and simultaneously blocks cell migration and endosome recycling thereby increasing cell surface CD4 and CXCR4. Thus, both active and inactive α1PI are sentinel to the fate of LDL receptor-coupled cargo.

Accordingly, in a first aspect, the invention features a method for determining the number of CD4+ T cells in a sample comprising determining the concentration of alpha 1 proteinase inhibitor (α1PI) in a sample, wherein the number of CD4+ T-cells is related to the concentration of α1PI.

In one embodiment, the sample is front a subject receiving PI therapy.

In another embodiment, the sample is from a subject not receiving PI therapy.

In another aspect, the invention features a method for determining the number of CD4+ T cells counts in a sample from a subject receiving PI therapy using formula I:

CD4 cells/μl=205+12α1PIμM (Formula I)

thereby determining the number of CD4+ T cells in the sample.

In still another aspect, the invention features a method for determining the number of CD4+ T cells counts in a sample from a subject not receiving PI therapy using Formula II:

CD4 cells/μl=−60+29α1PIμM (Formula II)

thereby determining the number of CD4+ T cells in the sample.

In still another aspect the invention features a method for determining the number of CD4+ T cells a sample comprising: determining the concentration of alpha 1 proteinase inhibitor (α1PI) in a sample and performing a calculation to determine from the concentration of α1PI in the sample the number of CD4+ cells in the sample; wherein the number of CD4+ T-cells is related to the concentration of at α1PI.

In one embodiment, the sample is from a subject receiving PI therapy.

In another embodiment, the sample is from a subject not receiving PI therapy.

In another embodiment, the concentration of α1PI is determined by measuring the optical density of the sample.

In another embodiment the concentration of α1PI is determined by quantitating the catalytic activity of α1PI.

In still another aspect, the invention features a method for determining the number of CD4+ T cells in a sample from a subject receiving PI therapy comprising: determining the concentration of α1PI in the sample and performing a calculation using Formula I:

CD4+ T cells/μl=205+12α1PIμM (Formula I)

thereby determining the number of CD-4+ T cells in the sample.

In still another aspect, the invention features a method for determining the number of CD4+ T cells in a sample from a subject not receiving PI therapy comprising: determining the concentration of α1PI in the sample and performing a calculation using Formula II:

CD4+ T cells/μl=−60+29α1PIμM (Formula II)

thereby determining the number of CD4+ T cells in the sample.

In still another aspect, the invention features a method for determining the number of CD4+ T cells in a sample from a subject comprising: determining the concentration of α1PI in the sample and performing a calculation using Formula III:

CD4+ T cells/μl=b(0)+b(1)×α1PIμM (Formula III)

thereby determining the number of CD4+ T cells in the sample.

In one embodiment, the subject is receiving PI therapy and b(0) is between 75 and 385 and b(1) is between 23 and 35.

In other embodiment, the subject is not receiving PI therapy and b(0) is between −200 and 50 and b(1) is between 25 and 35.

In another embodiment, α1PI is calculated as α1PI activity (OD₄₀₃nm).

In still another aspect, the invention features a method for determining the number of CD4+ T cells in a saliva sample from a subject comprising: determining the concentration of α1PI in the sample and performing a calculation using Formula IV:

Log [CD4 (cells/μl)]=b(0)+b(1)×Log [α1PI Index] (Formula IV)

thereby determining the number of CD4+ T cells in the sample.

In one embodiment, the α1PI Index is calculated as α1PI activity (OD₄₀₅ₙₘ)/serum concentration in saliva (OD₅₉₅ₙₘ).

In another embodiment, b(0) is between 2 and 3 and b(1) is between 0.2 and 0.3.

In another aspect the invention provides a method for determining the number of CD4+ T cells in a sample from a subject receiving PI therapy, comprising determining the concentration of α1PI in the sample, and determining the number of CD4+ T cells in the sample using a server comprising a memory storing an instruction set and data related to a plurality of formula defining the correlation between the concentration of α1PI and the number of CD4+ T cells in a sample, and a processor, whereby the processor determines the number of CD4+ T cells in the sample using Formula I

CD4+ T cells/μl=205+12α1PIμM (Formula I)

thereby determining the number of CD4+ T cells is the sample.

In another aspect, the invention provides a method for determining the number of CD4+ T cells in a sample from a subject receiving PI therapy, comprising determining the concentration of α1PI in the sample, and determining the number of CD4+ T cells using a server comprising a memory storing an instruction set and data related to a plurality of formula defining the correlation between the concentration of α1PI and the number of CD4+ T cells in a sample, and a processor, whereby the processor determines the number of CD4+ T cells in the sample using Formula II:

CD4+ T cells/μl=−60+29α1PIμM (Formula II)

thereby determining the number of CD4+ T cells in the sample.

In another aspect, the invention provides a method for determining the number of CD4+ T cells in a sample from a subject comprising determining the concentration of α1PI in the sample, and determining the number of CD4+ T cells in the sample using a server comprising a memory storing an instruction set and data related to a plurality of formula defining the correlation between the concentration of α1PI and the number of CD4+ T cells in a sample, and a processor, whereby the processor determines the number of CD4+ T cells in the sample using Formula III:

CD4+ T cells/μl=b(0)+b(1)×α1PIμM (Formula III)

thereby determining the number of CD4+ T cells in the sample.

In one embodiment, the subject is receiving PI therapy and b(0) is between 75 and 385 and b(1) is between 23 and 35.

In another embodiment, the subject is not receiving PI therapy and b(0) is between −200 and 50 and b(1) is between 25 and 35.

In another embodiment, α1PI is calculated as α1PI activity (OD₄₀₅nm).

In another aspect, the invention provides a method for determining the number of CD4+ T cells in a saliva sample, comprising determining the concentration of α1PI in the sample, and determining the number of CD4+ T cells in the sample using a server comprising a memory storing an instruction set and data related to a plurality of formula defining the correlation between the concentration of α1PI and the number of CD4+ T cells in a sample, and a processor, whereby the processor determines the number of CD4+ T cells in the sample using Formula IV:

Log [CD4 (cells/μl)]=b(0)+b(1)×Log [α1PI Index] (Formula IV)

thereby determining the number of CD4+ T cells in the sample.

In one embodiment, the α₁PI Index is calculated as α₁PI activity (OD₄₀₅ₙₘ)/serum concentration in saliva (OD₅₉₅ₙₘ).

In another embodiment, b(0) is between 2 and 3 and b(1) is between 0.2 and 0.3.

In one embodiment of anyone of the above aspects, the number of CD4+ T cells varies due to cycling. In a related embodiment, the cycling time is 14-27 days

In another embodiment of any one of the above aspects, the number of CD4+ T cells varies due to immunization, infection, changes in medication, malignancy, stress or immune failure.

In still another embodiment of any one of the above aspects, the method further comprises obtaining the sample from a subject.

In one embodiment of any one of the above aspects, the sample is from a subject receiving HIV protease inhibitor therapy. In one embodiment of any one of the above aspects, the sample is from a subject not receiving HIV protease inhibitor therapy.

In one embodiment of any one of the above aspects, the method is carried out in a microtiter plate.

In one embodiment of any one of the above aspects, the method is used to monitor a subject for a change in the number of CD4+ T cells, wherein a change in the number of CD4+ T cells indicates a need to start or change therapy.

In another aspect, the invention features a method for determining a course of treatment for a subject suffering from HIV according to the claimed methods, wherein a change in the number of CD4+ T cells is used to determine a course of treatment for the subject.

In one embodiment, the number of CD4+ T cells is greater than 200 CD4 cells/μl.

In another aspect, the invention features a method for determining treatment for a subject suffering from HIV or an α1PI deficiency disorder comprising determining the concentration of alpha 1 proteinase inhibitor (α1PI) in a sample; and calculating the number of CD4+ T-cells; wherein the number of CD4+ T cells in the sample determines the treatment for the subject.

In one embodiment, the subject is receiving PI therapy.

In another embodiment, the subject is not receiving PI therapy.

In another aspect, the invention features a method for determining treatment for a subject suffering from HIV or an α1PI deficiency disorder using Formula I:

CD4+ T cells/μl=205+12α1PIμM (Formula I)

wherein the number of CD4+ T cells determines the treatment for a subject suffering from HIV or an α1PI deficiency disorder.

In another aspect, the invention features a method for determining treatment for a subject suffering from HIV or α1PI deficiency disorder using formula II:

CD4+ T cells/μl=−60+29α1PIμM (Formula II)

wherein the number of CD4+ T cells determines the treatment for a subject suffering from HIV or an α1PI deficiency disorder.

In another aspect, the invention provides a method for determining a course of treatment for a subject suffering from HIV, comprising determining the number of CD4 T-cells according to any of the methods of the invention presented herein, wherein a change in the number of CD4+ T cells is used to determine a course of treatment for the subject.

In one embodiment, the number of CD4+ T cells is greater than 200 CD4 T cells/μl.

In another embodiment, the number of CD4+ T cells is less than 200 CD4 T cells/μl.

In another aspect the invention provides a method for determining treatment for a subject suffering from HIV or an α1PI deficiency disorder comprising: determining the number of CD4+ T-cells according to any the methods of the invention presented herein, wherein the number of CD4+ T cells in the sample determines the treatment for the subject.

In one embodiment, the subject is receiving PI therapy.

In another embodiment, the subject is not receiving PI therapy.

In another aspect, the invention provides a method for determining treatment for a subject suffering from HIV or an α1PI deficiency disorder comprising determining the concentration of α1PI in a sample derived from the subject and determining the number of CD4+ T cells in the sample by performing a calculation using Formula I:

CD4+ T cells/μl=205+12α1PIμM (Formula I)

wherein the number of CD4+ T cells determines the treatment for a subject suffering from HIV or an α1PI deficiency disorder.

In another aspect, the invention provides a method for determining treatment for a subject suffering from HIV or an α1PI deficiency disorder comprising determining the concentration of α1PI in a sample derived from the subject and determining the number of CD4+ T cells in the sample by performing a calculation using Formula II:

CD4+ T cells/μl=−60+29α1PIμM (Formula II)

wherein the number of CD4+ T cells determines the treatment for a subject suffering from HIV or an α1PI deficiency disorder.

In another aspect, the invention provides a method for determining treatment for a subject suffering from HIV or an α1PI deficiency disorder comprising determining the concentration of α1PI in a sample derived from the subject and determining the number of CD4+ T cells in the sample by performing a calculation using Formula III:

CD4+ T cells/μl=b(0)+b(1)×α1PIμM (Formula III)

wherein the number of CD4+ T cells determines the treatment for a subject suffering from HIV or an α1PI deficiency disorder.

In one embodiment, the subject is receiving PI therapy and b(0) is between 75 and 385 and b(1) is between 23 and 35.

In another embodiment, the subject is not receiving PI therapy and b(0) is between −200 and 50 and b(1) is between 25 and 35.

In another embodiment, α1PI is calculated as α1PI activity (OD₄₀₅nm).

In another aspect, the invention provides a method for determining treatment for a subject suffering from HIV or an a α1PI deficiency disorder comprising determining the concentration of α1PI in a saliva sample derived from the subject and determining the number of CD4+ T cells in the sample by performing a calculation using Formula IV:

Log [CD4 (cells/μl)]=b(0)+b(1)×Log [α1PI Index] (Formula IV)

wherein the number of CD4+ T cells determines the treatment for a subject suffering from HIV or an α1PI deficiency disorder.

In one embodiment, the α₁PI Index is calculated as α₁PI activity (OD₄₀₅ₙₘ)/serum concentration in saliva (OD₅₉₅ₙₘ)

In another embodiment b(0) is between 2 and 3 and b(1) is between 0.2 and 0.3. In one embodiment of any one of the above aspects, CD4+ T cell count varies due to cycling.

In another embodiment of any one of the above aspects, the α1PI deficiency disorder is a genetic disorder.

In one embodiment of any one of the above aspects, the treatment comprises HIV protease inhibitor therapy.

In a further embodiment, the cycling time is 14-27 days.

In another further embodiment of any one of the above aspects, the method further comprises obtaining the sample from a subject.

In one embodiment of any one of the above aspects, the number of CD4+ T cells varies due to immunization, infection, changes in medication, malignancy, stress or immune failure.

In still another embodiment of any one of the above aspects, the determination of α1PI concentration further comprises the steps of contacting the sample with a protease inhibited by α1PI and monitoring the catalytic activity of α1PI.

In a further embodiment, the protease inhibited by α1PI is porcine pancreatic elastase (PPE).

In one embodiment of any one of the above aspects, the concentration of α1PI is determined from total α1PI in the sample. In one embodiment of any one of the above aspects, the concentration of α1PI is determined from active α1PI in the sample.

In one particular embodiment of any one of the above aspect, the sample is from a subject receiving HIV protease inhibitor therapy.

In another embodiment of any one of the above aspects, the sample is from a subject not receiving HIV protease inhibitor therapy.

In a further embodiment of any one of the above aspects, the method is carried out in a microtiter plate.

In another embodiment of any one of the above aspects, the number of CD4+ T cells is greater than 200 CD4 cells/μl.

In another embodiment of any one of the above aspects, the number of CD4+ T cells is less than 200 CD4 cells/μl.

In another particular embodiment, the invention features a method for diagnosing a subject with HIV or an α1PI deficiency disorder, comprising determining the number of CD4+ T cells in the subject by the method of any one of the aspects described herein.

In one embodiment of any one of the above aspects, a level of α₁PI less than 20 μM indicates the subject is suffering from HIV.

In one embodiment of any one of the above aspects, a level of α₁PI less than 20 μM indicates the subject is in need of HIV antiviral therapy.

In another further embodiment of any one of the above aspects, the subject is a human.

In still another embodiment of any one of the above aspects, the sample is selected from the group consisting of: blood serum, blood plasma, and saliva.

In still another embodiment of any one of the above aspects, said prediction interval is 95% as compared to flow cytometric methods of determining the number of CD4+ T cells in a sample.

In still another embodiment of any one of the above aspects, the calculation is performed in a spreadsheet with mathematical capabilities.

In still another embodiment of any one of the above aspects the calculation is performed by a software macro.

In still another embodiment of any one of the above aspects, the calculation is performed by a computer.

In another aspect, the present invention features a kit for determining the number of CD4+ T cells in a sample according to the method of any one of the aspects described herein, and instructions for use.

In another aspect, the invention features a kit for determining the number of CD4+ T cells in a sample comprising a reagent for determining the concentration of α1PI in a sample, and instructions for use to determine the number of CD4+ T cells in a sample.

In one embodiment, the reagent is a protease inhibited by α1PI.

In another embodiment, the number of CD4+ T cells in a sample is determined using Formula I:

CD4+ T cells/μl=205+12α1PIμM (Formula I).

In another preferred embodiment, the number of CD4+ T cells in a sample is determined using Formula II:

CD4+ T cells/μl=−60+29α1PIμM (Formula II).

In another further embodiment, the kit further comprises one or more antiretroviral agents.

In another embodiment, the number of CD4+ T cells In a sample is determined by determining the concentration of α1PI in the sample and performing a calculation using Formula I:

CD4+ T cells/μl=205+12α1PIμM (Formula I)

thereby determining the number of CD4+ T cells in the sample.

In another embodiment the number of CD4+ T cells in a sample is determined by determining the concentration of α1PI in the sample and performing a calculation using Formula II:

CD4+ T cells/μl=−60+29α1PIμM (Formula II)

thereby determining the number of CD4+ T cells in the sample.

In another embodiment, the number of CD4+ T cells in a sample is determined by determining the concentration of α1PI in the sample and performing a calculation using Formula III:

CD4+ T cells/μl=b(0)+b(1)×α1PIμM (Formula III)

thereby determining the number of CD4+ T cells in the sample.

In another embodiment, the number of CD4+ T cells in a sample is determined by determining the concentration of α1PI in the sample and performing a calculation using Formula IV:

Log [CD4 (cells/μl)]=b(0)+b(1)×Log [α₁PI Index] (Formula IV)

thereby determining the number of CD4+ T cells in the sample.

In another aspect, the invention provides a server for facilitating a diagnostic tool, wherein the server calculates the number of CD4+ T cells in a sample and wherein the server comprises:

(a) a memory storing an instruction set and data related to a formula defining the correlation between the concentration of alpha 1 proteinase inhibitor and the number of CD4+ T cells in a sample; and

(b) a processor for running the instruction set, the processor being in communication with the memory, wherein the processor is operative to calculate the number of CD4+ T cells in the sample from the concentration of alpha 1 PI and wherein the formula is

CD4+ T cells/μl=205+12α1PIμM (Formula I)

In another aspect, the invention provides a server for facilitating a diagnostic tool, wherein the server calculates the number of CD4+ T cells in a sample and wherein the server comprises:

(a) a memory storing an instruction set and data related to a formula defining the correlation between the concentration of alpha 1 proteinase inhibitor and the number of CD4+ T cells in a sample; and

(b) a processor for running the instruction set, the processor being in communication with the memory, wherein the processor is operative to calculate the number of CD4+ T cells in the sample from the concentration of alpha 1 PI and wherein the formula is

CD4+ T cells/μl=−60+29α1PIμM (Formula II).

In another aspect, the invention provides a server for facilitating a diagnostic tool, wherein the server calculates the number of CD4+ T cells in a sample and wherein the server comprises:

(a) a memory storing an instruction set and data related to a formula defining the correlation between the concentration of alpha 1 proteinase inhibitor and the number of CD4+ T cells in a sample; and

(b) a processor for running the instruction set, the processor being in communication with the memory, wherein the processor is operative to calculate the number of CD4+ T cells in the sample from the concentration of alpha1 PI and wherein the formula is

CD4+ T cells/μl=b(0)+b(1)×α1PIμM (Formula III)

In another aspect, the invention provides a server for facilitating a diagnostic tool, wherein the server calculates the number of CD4+ T cells in a sample and wherein the server comprises;

(a) a memory storing an instruction set and data related to a formula defining the correlation between the concentration of alpha 1 proteinase inhibitor and the number of CD4+ T cells in a sample; and

(b) a processor for running the instruction set, the processor being in communication with the memory, wherein the processor is operative to calculate the number of CD4+ T cells in the sample from the concentration of alpha 1 PI and wherein the formula is

Log [CD4 (cells/μl)]=b(0)+b(1)×Log [α₁PI Index] (Formula IV)

In another aspect the invention provides a method for determining the number of CD4+ T cells in a sample comprising: subjecting the sample to an elastase-specific peptide substrate and transforming the concentration measurement of α1PI in to sample to a measurement of the number of CD4+ T cells in said sample by using any one of Formula I, II, III or IV.

In one embodiment of any one of the aspects of the invention, the method of determining the number of CD4+ T cells accounts for CD4+ T cell cycling.

In another aspect, the invention provides a method for determining the number of CD4+ T cells in a sample comprising (i) serially diluting the sample; (ii) contacting the sample with a colorimetric elastase-specific peptide substrate; and (iii) measuring a colorimetric change in the sample after the step of contacting the sample with the elastase-specific peptide substrate.

In another aspect the invention provides a method for determining the number of CD4+ T cells in a sample comprising determining the concentration of α1PI in the sample and performing a calculation using Formula III, where for patients subjected to antiretroviral therapeutic agents b(0) is 205 and b(1) is 12; where for patients not yet subjected to antiretroviral therapeutic agents b(0) is −60 and b(1) is 29.

In one embodiment, b(0) is a number between 75 and 385 inclusive and b(1) is a number between 23 and 35 inclusive.

In another embodiment, b(0) is a number between −200 and 50 inclusive and b(1) is a number between 25 and 35 inclusive.

In another embodiment, b(0) is a number between −200 and 50 inclusive and b(1) is a number between 25 and 35 inclusive.

In another embodiment, b(0) is a number between −1000 and 1000 inclusive for example, −1000 and 500, −1000 and 400, −1000 and 300, −1000 and 200, −1000 and 100, −500 and 1000, −400 and 1000, −300 and 1000, −200 and 1000, −500 and 500, −400 and 400, −300 and 300, −200 and 200 and b(1) is a number between 1 and 1000 inclusive, for example 1 and 100 inclusive or 1 and 50 inclusive or 1 and 10 inclusive; where b(0) and b(1) are determined from a sample of patient populations.

In another aspect the invention provides a method for determining the number of CD4+ T cells in a sample comprising determining the concentration of α1PI in the sample and performing a calculation using Formula IV, where for patients subjected to antiretroviral therapeutic agents b(0) is between 2 and 3 inclusive and b(1) is between 0.2 and 0.3 inclusive.

In one embodiment, b(0) is between 1 and 1000 inclusive or 1 and 100 inclusive or 1 and 50 inclusive or 1 and 10 inclusive and b(1) is between 0.1 and 1000 inclusive or 0.1 and 100 inclusive or 0.1 and 50 inclusive or 0.1 and 10 inclusive or 0.1 and 1 inclusive.

Other aspects of the invention are described in or are obvious from the following disclosure, and are within the ambit of the invention.

### BRIEF DESCRIPTION Of THE DRAWINGS

The following Detailed Description, given by way of example, but not intended to limit the invention to specific embodiments described, may be understood in conjunction with the accompanying drawings, incorporated herein by reference. Various preferred features and embodiments of the present invention will now be described by way of non-limiting example and with reference to the accompanying drawings in which:

FIG. 1 is a graph that shows active α1PI is rate limiting for CD4+ lymphocytes in HIV-1 disease. In 34 HIV-1 patients studies, 26 were above 200 and 8 were below 200 CD4 cells/μl at the time of blood collection. All patients were measured for HIV RNA copies/ml, CD4, CXCR4, CCR5, CXCL12 levels, active and inactive α1PI. In the 26 patients with <200 CD4 cells/μl, there was no relationship between CD4+ lymphocyte levels and active or inactive α1PI. Patients receiving HIV-1 Protease Inhibitor therapy (PI) are depicted by squares. All other patients are depicted by circles.

FIG. 2 is a graph that shows prediction intervals for CD4+ lymphocytes. Patients depicted in FIG. 1 with undetectable viral load are partitioned into two groups, those on HIV Protease Inhibitor (PI) therapy and those not on PI therapy. The regression line for CD4 T cell count in patients on PI therapy (CD4 cells/μl=205+12 α1PI μM), r2=0.69, n=15) differs from patients not on PI therapy (CD4 cells/μl=−60+29 α1PI μM), r2=0.74, n=11). The 95% confidence intervals for each regression line are depicted in dark gray. The 95% prediction intervals are depleted in light gray. The prediction algorithms were validated using 8 additional HIV-1 patients and all predictions fell within the relevant 95% prediction intervals.

FIG. 3(a-c) is a panel of graphs that show accuracy of predicting CD4 T cell counts front α1PI. a) Computer generated sine wave analysis of CD4 cycling was used to calculate the peak-to-peak amplitude and axis of oscillation in 5 patients. Amplitude represents CD4 variation due to cycling. The CD4 axis of oscillation is an unvarying constant that represents the best estimate of CD4 T cell count. b) Regression analysis of amplitude versus axis of oscillation yields a linear relationship (amplitude=−8+0.19*axis of oscillation, r^{2=0.98}). c) The linear relationship derived in part b) was used to calculate the expected amplitude of points along the regression lines from FIG. 2. Expected peak-to-peak amplitude (dotted lines) for points along the regression lines (center lines) from FIG. 2 are depicted alongside the 95% confidence intervals (surrounding solid lines) from the same regression lines from FIG. 2. The expected amplitude overlaps with the confidence intervals except at exceedingly low values. The overlapping is interpreted to mean that there is 95% confidence that the reason the actual CD4 T cell counts do not fall exactly onto the regression lines in FIG. 2 is due to CD4 cycling.

FIG. 4(a and b) are two graphs that show corresponding cyclic variation in blood cells, α1PI, and viral load in patients treated with α1PI augmentation. Baseline CD4+ lymphocyte levels were determined in patients Alpha, Seta, and Gamma to be 257, 276, and 148 cells/pl, respectively. Blood was collected prior to infusion, and each data point represents patient status at 7 days post-infusion such that wk 9 represents patient status after the 8th wk of treatment. (a) CD4+ lymphocytes, CD4/CD8 ratios, and CD4% () vs. the corresponding CD8% (◯) are presented with respect to months of disease diagnosis. Shaded areas represent normal reference ranges for CD4, CD4/CD8 ratio, and CD4%. Black arrows designate initiation of ZEMAIRA treatment. White arrows designate initiation of antiretroviral therapy, (b) Patients Alpha, Beta, Gamma, and PIZZ-1 were monitored weekly for changes in blood cell subtypes and serum levels of α1PI HIV-1+ patients were monitored for changes in HIV RNA. Treatment wk 0 represents baseline pre-treatment values. In some instances, blood samples were not acceptable for measuring blood cells, HIV RNA, or α1PI due to delay in sample delivery or hemolysis, and these are depicted as gaps in the line graphs.

FIG. 5 are two graphs that show duration of increase in CD4+ lymphocytes following α1PI therapy. Comparison of the change in CD4+ lymphocytes represented in FIG. 1 before (), during (), and after (*) α1PI augmentation therapy demonstrates that the duration of benefit is 1 or 2 weeks post-treatment (Patient Beta), but not 5 or 14 weeks post-treatment (Patient Alpha). Linear regression of CD4+ lymphocyte changes before (solid line) and during (dashed line) show significant improvement.

FIG. 6(a and b) are graphs that show functional characteristics of expanded CD4+ lymphocytes, a) NFκB phosphorylation in response to stimulation of the T cell antigen receptor complex (light gray) as compared to unstimulated cells (dark gray). Isolated CD4+ T cells were cultured at 10⁶ cells/μl for 3 days in the presence of antibodies reactive with CD2, CD3, and CD28 as described in Methods. Cells from patient Alpha were isolated after 11 weeks of ZEMAIRA therapy. Cells from patient Delta were isolated at baseline. Culture supernatants were simultaneously measured for cytokine release (Table 2). b) Percentage of immature, naïve, and memory T cells. Analysis for the presence of naïve (CD4+CD45RA+) and memory (CD4+CD45RO+) T cells in whole blood was performed as described in Methods. Phenotypic analysis was performed at 3-8 different time points during therapy using blood collected from patients depicted left to tight as follows: Alpha, Beta, Gamma, Delta, PI_{ZZ}-1, and PI_{ZZ}-2. Values represent mean and standard deviation. Stars (*) indicate statistical differences with respect to non-HIV-1 control values, p<0.001 and power of test a >0.99.

### DETAILED DESCRIPTION OF THE INVENTION

### 1. Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed. 1988); The Glossary of Genetics, 5th Ed., R. Rieger et al. (eds.), Springer Verlag (1991); and Hale & Marham. The Harper Collins Dictionary of Biology (1991). As used herein, the following terms have the meanings ascribed to them unless specified otherwise.

As used, in the specification and claims, the singular form “a”, “an” and “the” include plural references unless the contest clearly dictates otherwise.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Unless specifically stated or obvious from context, as used herein, the term “or” is understood to be inclusive.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof. Any compositions or methods provided herein can be combined with one or more of any of the other compositions and methods provided herein.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

The terms “administration” or “administering” are defined to include an act of providing a compound or pharmaceutical composition of the invention to a subject in need of treatment. In the instant invention, preferred routes of administration include parenteral administration, preferably, for example by injection, for example by intravenous injection.

As used herein, the term “control” is meant a standard or reference condition.

The term “α-test” as used herein is meant to refer to a test that determines CD4 enumeration based on α1PI concentration.

The term “active α1PI” as used herein is meant to refer to the fraction of α1PI in plasma or other fluids that has the capacity to inhibit elastase activity.

The term “inactive α1PI” as used herein is meant to refer to the fraction of α1PI in plasma or other fluids that does not have the capacity to inhibit elastase activity. Active α1PI may be inactivated by proteolytic cleavage, proteinase complexing, antibody complexing, or oxidation.

The term “human immunodeficiency virus” or HIV is mean to refer to a virus that consists of either an HIV-1 or an HIV-2 virus, and more particularly any virus strain or isolate of an HIV-1 or an HIV-2 virus.

As used herein, the term “alpha 1-Proteinase inhibitor” (α1PI) is meant to refer to a glycoprotein produced by the liver and secreted into the circulatory system. α1PI belongs to the Serine Proteinase Inhibitor (Serpin) family of proteolytic inhibitors. This glycoprotein of MW of 50,600 Da consists of a single polypeptide chain containing one cysteine residue and 12-13% carbohydrates of the total molecular weigh. α1PI has three N-glycosylation sites at asparagine residues 46, 83 and 247, which are occupied by mixtures of complex bi- and triantennary glycans. This gives rise to multiple α1PI isoforms, having isoelectric point in the range of 4.0 to 5.0. The glycan monosaccharides include N-acetylglucosamine, mannose, galactose, fucose and sialic acid. α1PI serves as a pseudo-substrate for elastase, elastase attacks the reactive center loop of the α1PI molecule by cleaving the bond between methionine358-serine359 residues to form an α1PI-elastase complex. This complex is rapidly removed from the blood circulation. α1PI is also referred to as “alpha-1 antitrypsin” (AAT). In certain embodiments, α1PI is human α1PI and is encoded by the amino acid sequence set forth by NCBI Accession No. KO1396.

As used herein, the term “subject” is intended to include vertebrates, preferably a mammal. Mammals include, but are not limited to, humans.

It should be appreciated that the subject technology can be implemented and utilized in numerous ways, including without limitation as a process, an apparatus, a system, a device, a method for applications now known and later developed or a computer readable medium. These and other unique features of the system disclosed herein will become more readily apparent from the following description and the accompanying drawings.

A computer or server means one or more digital data processing devices used in connection with various embodiments of the invention. Such a device generally can be a personal computer, computer workstation (e.g., Sun, HP), laptop computer, server computer, mainframe computer, handheld device (e.g., personal digital assistant, Pocket PC, cellular telephone, etc.), information appliance, printed circuit board with components or any other type of generic or special-purpose, processor-controlled device capable of receiving, processing, displaying, and/or transmitting digital data. A typical computer includes random access memory (RAM), mechanisms and structures for performing I/O operations, a storage medium such as a magnetic hard disk drive(s), and an operating system (e.g., software) for execution on the central processing unit. The computer also has input and output devices such as a keyboard and monitor, respectively.

A processor generally is logic circuitry that responds to and processes instructions that drive a computer and can include, without limitation, a central processing unit, an arithmetic logic unit, an application specific integrated circuit, a task engine, and/or any combinations, arrangements, or multiples thereof.

Software or code generally refers to computer instructions which, when executed on one or more digital data processing devices, cause interactions with operating parameters, sequence data/parameters, database entries, network connection parameters/data, variables, constants, software libraries, and/or any other elements needed for the proper execution of the instructions, within an execution environment in memory of the digital data processing device(s).

A module is a functional aspect, which may include software and/or hardware. Typically, a module encompasses the necessary components to accomplish a task. It is envisioned that the same hardware could implement a plurality of modules and portions of such hardware being available as needed to accomplish the task. Those of ordinary skill will recognize that the software and various processes discussed herein are merely exemplary of the functionality performed by the disclosed technology and thus such processes and/or their equivalents may be implemented in commercial embodiments in various continuations without materially affecting the operation of the disposed technology.

Other definitions appear in context throughout the disclosure.

### METHODS OF THE INVENTION

It has been found by the present invention that the number of CD4+ T lymphocytes in blood is driven by the concentration of the protein α1PI, and the linear relationship between α1PI and CD4+ lymphocytes, measured in serum or saliva, allows the use of a mathematical formula to produce accurate CD4 T cell counts. Accordingly, the present invention describes, for the first time, a low-cost cell-free assay, the α-test, that provides point-of-care enumeration of CD4+ T lymphocytes in the blood of HIV patients.

The present invention features methods for determining the CD4+ T cell counts in a sample.

In preferred aspects, the present invention features method for determining the number of CD4+ T cells in a sample comprising determining the concentration of alpha 1 proteinase inhibitor (α1PI) in a sample, wherein the number of CD4+ T-cells is related to the concentration of α1PI.

In clinical practice, symptomatology and measurements of immune function, notably levels of CD4+ T lymphocytes, are used to guide the treatment of HIV-infected persons, A CD4 count of <=200 cells/μl has been included as an AIDS-defining event according to the Centers for Disease Control, as these measurements are useful predictors for the onset of opportunistic diseases such as Pneumocystis carinii pneumonia. With the advent of highly active antiretroviral therapy, CD4 T-lymphocyte measurements have been used to monitor immune reconstitution (Autran et al., 1997, Science 1997;277:112-116).

The current predicate methodology for determining absolute CD4 T-lymphocyte counts is dependent upon immunophenotypic identification of cells with fluorescently labelled monoclonal antibodies directed against the CD4 antigen. Relative percentages of CD4 T cells are determined with a flow cytometer. An absolute CD4 count is derived by multiplying the percentage of lymphocytes that are CD3+ CD4+ by the absolute lymphocyte count determined with a hematology instrument.

However, factors other than HIV can affect CD4 count, including infections, time of day, smoking, stress and which lab tests the blood sample. For instance, overnight shipment of blood may result in increased intrinsic variability in the absolute lymphocyte count depending on the hematology instrument that is used.

Accordingly, the present invention features methods for determining the CD4+ T cell count in a sample, wherein the number of CD4+ T-cells is related to the concentration of α1PI.

Alpha-1 proteinase inhibitor (α1PI) is a derivative of human plasma belonging to the family of serine proteinase inhibitors. It is a glycoprotein having an average molecular weight of 50,600 daltons, produced by the liver and secreted into the circulatory system. The protein is a single polypeptide chain, to which several oligosaccharide units are covalently bound. α1PI has a role in controlling tissue destruction by endogenous serine proteinases, and is the most prevalent serine proteinase inhibitor in blood plasma. Among others, α1PI inhibits trypsin, chymotrypsin, various types of elastases, skin collagenase, renin, urokinase and proteases of polymorphonuclear lymphocytes.

The normal role of α1PI is to regulate the activity of leukocyte elastase, which breaks down foreign proteins present in the lung. When α1PI is not present in sufficient quantities to inhibit elastase activity, the elastase breaks down lung tissue. In time, this imbalance results in chronic lung tissue damage and emphysema. α1PI is currently used therapeutically for the treatment of pulmonary emphysema in patients who have a genetic deficiency in α1PI. Purified α1PI has been approved for replacement therapy in these patients.

In a preferred embodiment of the invention, the method further comprises calculating the coordinates of the intersection of two linear lines by regression analysis so determine the concentration of α1PI, thereby determining CD4+ T cell counts in the sample.

In another embodiment, the CD4+ T cell count varies due to cycling.

The standard methods for monitoring CD4 T cell counts have not previously taken into account the 23-day cyclic changes. Thymopoiesis in humans has not previously been characterized, but in adult mice thymopoiesis is a multi-step process that is highlighted by a 21-day cycle of coordinated journeying of progenitor cells between adult bone marrow and thymus (Donskoy et al., 2003). In certain embodiments, the cycling time is 14-27 days. In other embodiments, the cycling time is 23 days.

CD4+ T cell count can vary due to immunization, infection, changes in medication, malignancy, stress or immune failure.

The computational method for CD4 enumeration is due to the linear relationship between active α1PI and CD4 T cell counts. The well-known effect of HIV protease inhibitor therapy to increase CD4 T cell counts may, in part, result from effects that simulate the physiologically relevantα1PI effect. Accordingly, the method for CD4 enumeration described herein incorporates CD4 cycling in establishing CP4 T cell reference ranges. Since CD4 T cells exhibit sinusoidal cycling, the axis of oscillation about which the CD4 T cells cycle allows an accurate and precise measurement of CD4 T cell counts. The methods described herein measure active α1PI in a sample, for example in serum or saliva, and calculates the CD4 T cell axis of oscillation.

In preferred embodiments, in the method of the invention described herein, the step of calculating the number of CD4+ T cells further comprises determining an axis of oscillation for CD4+ T cell cycling by sine curve analysis, thereby determining CD4+ T cell counts in the sample. In other further embodiments, the step of calculating the number of CD4+ T-cells further comprises determining a peak-to-peak amplitude for CD4+ T cell cycling by sine curve analysis, thereby determining CD4+ T cell counts in the sample.

Preferably, the axis of oscillation is correlated with the peak-to-peak amplitude in a linear relationship.

Using the method described herein, in preferred embodiments, the determination of α1PI concentration further comprises the steps of contacting the sample with a protease inhibited by α1PI or another protease inhibited by α1PI and monitoring the catalytic activity of α1PI.

In exemplary embodiments of the invention, monitoring the catalytic activity of α1PI further comprises the steps of preparing a plurality of serial dilutions of the sample, incubating the dilutions with varying concentrations of a protease inhibited by α1PI, monitoring the catalytic activity, and calculating the coordinates of the intersection of two linear lines by regression analysis, thereby monitoring the catalytic activity of α1PI.

Preferably, the protease inhibited by α1PI is porcine pancreatic elastase (PPE).

In complex mixtures, α1PI competes for binding to PPE with other proteinase inhibitors or ligands present in the mixture. For example, PPE has higher affinity for α2macroglobulin (α2M) than for α1PI, and when complexed with α2M. PPE retains the ability to cleave small substrates. In the presence of α2M, PPE binds α2M and is protected from inhibition by α1PI, and the complexation of PPE with α2M can be measured by detecting the activity of PPE using the elastase-specific substrate succinyl L-Ala-L-Ala-L-Ala-p-nitroanalide (SA3NA). To measure the inhibitory capacity of α1PI in complex mixtures such as serum, two-fold serial dilutions of serum are incubated with a constant, saturating, concentration of PPE. The added PPE is bound by β2M and α1PI in the diluted serum dependant on their concentrations, the greater the concentration of serum, the greater the concentration of α2M and α1PI. Since there is more α1PI in serum than α2M, as serum is diluted, α2M is diluted out, and in the absence of α2M, PPE is bound and inhibited by α1PI. The complexation of PPE with α1PI can be measured by detecting the loss of activity of PPE using SA3NA. As serum is further diluted, α1PI is also diluted out, and the loss of complexation of PPE with α1PI can be measured by detecting the gain in activity of PPE using SA3NA. The plot of PPE activity versus serum dilution makes a V shaped curve, PPE activity first decreasing as serum is diluted, and then increasing as serum is further diluted. The nadir of PPE activity is used to calculate the precise concentration of active α1PI in the mixture.

Accordingly, in the described method, one of the two lines is formed by the sample concentration and the other line is formed by residual catalytic activity. Further, the catalytic activity decreases linearly in relation to the dilutions of the sample to a minimum point, and then increases in relation to the dilution of the sample.

In certain embodiments, active α1PI is meant to refer to the fraction of α1PI in plasma or other fluids that has the capacity to inhibit elastase activity. In other embodiments, inactive α1PI is meant to refer to the fraction of α1PI in plasma or other fluids that does not have the capacity to inhibit elastase activity. Active α1PI may be inactivated by proteolytic cleavage, proteinase completing, antibody complexing, or oxidation.

In certain embodiments, the concentration of α1PI is determined from total α1PI in the sample. In other embodiments, the concentration of α1PI is determined from active α1PI in the sample.

The method can be standardized by measuring the maximum protease added to the diluted samples, wherein the protease activity detected in the presence of serum is divided by the maximum protease added, thereby standardizing to the maximum protease added.

### Monitoring and Diagnostics

The methods described herein for determining the CD4+ T cell counts in a sample can be used to monitor a subject for a change in CD4+ T cell number, wherein a change in CD4+ T cell number indicates a need to start or change therapy.

Such an embodiment may be used for assessing the risk of AIDS, as well as the onset of opportunistic diseases such as Pneumocystis carinii pneumonia. Counting of CD4+ T-helper/inducer cells may also be used as a gauge of immune reconstitution following treatment with anti-HIV drugs.

According to certain embodiments of the invention, CD4+ T cell number is used to determine the course of treatment for the subject.

The method can be used to determine the treatment for a subject suffering from HIV or an α1PI deficiency disorder.

Full length active α₁proteinase inhibitor (α₁PI, α₁antitrypsin) is composed of 394 amino acids (aa) having a mass of approximately 55 kDa when fully glycosylated. Hepatocytes are the primary source of α₁PI, and in normal, healthy individuals, the range of circulating α₁PI is 20-53 μM between the 5^{th} and 95^{th} percentiles. However, during the acute phase of the inflammatory response, α₁PI may increase as much as 4-fold to 200 μM. There are four common alleles of α₁PI, and these are synthesized and secreted principally by hepatocytes. However, there are more than a hundred genetic variants, some of which produce misfolded molecules that prohibit secretion, e.g. the Z allele. Individuals with this inherited form of α₁PI deficiency manifest with 10-15% of the normal level of α₁PI in blood. Affected individuals, especially males, are notably susceptible to respiratory infections and emphysema, and 80% who survive to adulthood succumb to respiratory failure between the fourth and sixth decades of life. Prevalence is 0.03%, and α₁PI augmentation therapy in affected individuals is the only approved therapeutic application of α₁PI. Thus, in certain embodiments, the α1PI deficiency disorder is a genetic disorder.

Accordingly, the method comprises determining the concentration of alpha 1 proteinase inhibitor (α1PI) in a sample, calculating the number of CD4+ T-cells from the concentration of α1PI, wherein the number of CD4+ T cell counts in the sample determines the treatment for the subject.

HIV infection/AIDS, is characterized by a decline in the number of CD4+ T cells. HIV infections progress through a number of different clinical stages which may be distinguished in a variety of ways. The U.S. Centers for Disease Control and Prevention (CDC) classification system and the World Health Organization (WHO) Clinical Staging and Disease Classification System are two known systems for classifying progression of HIV/AIDS. The CDC disease staging system (last revised in 1993) assesses the severity of HIV disease by CD4 cell counts and by the presence of specific HIV-related conditions. The definition of AIDS includes all HIV-infected individuals with CD4 counts of <200 cells/μL (or CD4 percentage <14%) as well as those with certain HIV-related conditions and symptoms. Although the fine points of the classification system rarely are used in the routine clinical management of HIV-infected patients, a working knowledge of the staging criteria (in particular the definition of AIDS) is useful in patient care. In addition, the CDC system is used in clinical and epidemiologic research.

In contrast to the CDC system, the WHO Clinical Staging and Disease Classification System (revised in 2005) can be used readily in resource-constrained settings without access to CD4 cell count measurements or other diagnostic and laboratory testing methods. The WHO system classified HIV disease on the basis of clinical manifestations that can be recognized and treated by clinicians in diverse settings, including resource-constrained settings, and by clinicians with varying levels of HIV expertise and training.

For example, in certain embodiments, the invention describes a method for determining treatment for a subject suffering from HIV or an α1PI deficiency disorder comprising determining the concentration of alpha 1 proteinase inhibitor (α1PI) in a sample, and calculating the number of CD4+ T-cells from the concentration of α1PI, where the number of CD4+ T cell counts in the sample determines the treatment for the subject.

Preferably, the method further comprises calculating the coordinates of the intersection of two linear lines by regression analysis to determine the concentration of α1PI, thereby determining CD4+ T cell counts in the sample.

In further steps of the method, the step of calculating the number of CD4+ T-cells further comprises determining an axis of oscillation for CD4+ T cell cycling by sine curve analysis, thereby determining CD4+ T cell counts in the sample. In other steps of the method, the step of calculating the number of CD4+ T-cells further comprises determining a peak-to-peak amplitude for CD4+ T cell cycling by sine curve analysis, thereby determining CD4+ T cell counts in the sample. Preferably, the axis of oscillation is correlated with the peak-to-peak amplitude in a linear relationship.

The methods and compositions described herein may be also used to monitor health status and to determine the presence of latent infections, for example. The methods and compositions described herein may also be used to monitor the progress of treatment, or to provide for an indication of the prognosis.

As described herein, in any of the described methods, it is a finding of the present invention that CD4+ T cell count varies due to cycling.

In various preferred embodiments of the invention, the methods are used to diagnose, detect or monitor HIV or anα1PI deficiency disorder.

The treatment may comprise HIV protease inhibitor therapy.

In the methods described, the subject preferably has a CD4+ T cell number that is greater thank 200 CD4 cells/μl.

In the methods described, the subject preferably has a level of α₁PI less than 20 μM indicating the subject is suffering from HIV.

The methods described may be advantageously employed in determining a course of treatment, in the diagnosis of diseases, in particular HIV or an α1PI deficiency disorder, through determining the number of CD4+ T cell counts in a sample from the subject.

The diagnostic methods described herein may preferably further comprise determining the quantity of CD4+ cells in the subject by the methods described herein.

### Subjects and Samples

In any of the methods as described herein, the subject may be a mammal. Preferably, the subject is a human.

The invention features methods where the subject is a human subject that is suffering from HIV. In certain embodiments of the invention, the sample is from a subject receiving HIV protease inhibitor therapy. In other embodiments of the invention, the sample is from a subject not receiving HIV protease inhibitor therapy.

HIV protease inhibitor therapy may be one or more antiretroviral drugs. Antiretroviral drugs inhibit the replication of HIV. When antiretroviral drugs are given in combination, e.g. one or more, two or more, HIV replication and immune deterioration can be delayed, and survival and quality of life improved. Taking two or more antiretroviral drugs at a time is called combination therapy. Taking a combination of three or more anti-HIV drugs is sometimes referred to as Highly Active Antiretroviral Therapy (HAART). There are over 20 approved antiretroviral drugs although all are licensed or available in every country. Antiretroviral drug classes include: Nucleoside/Nucleotide Reverse Transcriptase Inhibitors (NRTI), Non-Nucleoside Reverse Transcriptase Inhibitors (NNRTI), Protease Inhibitors, Fusion or Entry Inhibitors, and Integrase Inhibitors.

For example, a common drug combination given to those beginning treatment consists of two NRTIs combined with either an NNRTI or a “boosted” protease inhibitor. Ritonavir (in small doses) is most commonly used as the booster; it enhances the effects of other protease inhibitors so they can be given in lower doses. An example of a common antiretroviral combination is the two NRTIs zidovudine and lamivudine, combined with the NNRTI efavirenz.

The number of CD4+ T cells can be measured in different types of samples. Preferably, the sample is a blood sample that is obtained from the subject. The blood sample can be a blood serum sample or a blood plasma sample. In other embodiments, the sample is a saliva sample that is obtained from the subject.

The methods of the invention as described herein can suitably be carried out in a microtiter plate, for example a plate with 6, 24, 96, or more wells. In exemplary embodiments, the microtiter plate is an 8 well plate. Microtiter plate are suitably sized to conform to automated processors, dispensers, washers or readers known in the art to facilitate reagent addition, microplate replication, serial dilution and detection applications.

### Kits

The present invention also provides kits, for example kits for determining CD4+ T cell counts in a sample comprising a protease inhibited by α1PI for monitoring the catalytic activity of α1PI and instructions for determining the concentration of alpha 1 proteinase inhibitor (α1PI) in a sample and calculating the number of CD4+ T-cells from the concentration of α1PI to determine CD4+ T cell counts in the sample.

In one aspect, the invention features a kit for determining the number of CD4+ T cells in a sample comprising a reagent for determining the concentration of α1PI in a sample, and instructions for use to determine the number of CD4+ cells in a sample.

In one embodiment, the reagent is a protease inhibited by α1PI.

In another embodiment, the number of CD4+ T cells in a sample is determined using Formula I:

CD4+ T cells/μl=205+12α1PIμM (Formula I).

In another embodiment, the number of CD4+ T cells ha a sample is determined using Formula II:

CD4+ T cells/μl=−60+29α1PIμM (Formula II).

In another embodiment, the number of CD4+ T cells in a sample is determined using Formula III:

CD4+ T cells/μl=b(0)+b(1)×α1PIμM (Formula III).

In another embodiment, the number of CD4+ T cells in a sample is determined using Formula IV:

Log [CD4 (cells/μl)]=b(0)+b(1)×Log [α₁PI Index] (Formula IV).

In a further embodiment, the kit further comprises one or more antiretroviral agents.

The kit may be used in determining the treatment for a subject suffering from HIV or an α1PI deficiency disorder.

Thus, for example, a kit may be relevant to HIV monitoring, determining patient treatment, diagnosis or detection may comprise a container with a protease inhibited by α1PI, for example PPE. The kit is suitable for determining CD4 counts and may also comprise one or more anti-retroviral drugs for example, such as protease inhibitors, AZT, etc.

In certain embodiments, the kits comprises a sterile container, for example boxes, ampules, bottles, vials, tubes, bags, pouches, blister-packs, or other suitable container form known in the art. Such containers can be made of plastic, glass, laminated paper, metal foil, or other materials suitable for holding nucleic acids. The instructions will generally include information about the use of the kit according to the methods as described herein. In other embodiments, the instructions include at least one of the following: methods for using the enclosed materials for the treatment or prevention of AIDS; precautions; warnings; indications; clinical or research studies; and/or references. The instructions may be printed directly on the container (when present), or as a label applied to the container, or as a separate sheet, pamphlet, card, or folder supplied in or with the container.

The following examples are offered by way of illustration, not by way of limitation. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

### EXAMPLES

### Example 1

### The α-Test

The principle of the α-test for CD4 enumeration described herein is the quantitation of the active concentration of α1PI in serum (U.S. Pat. No. 6,887,678, incorporated by reference in its entirety herein) by measuring its inhibition of excess porcine pancreatic elastase (PPE, Sigma-Aldrich). The decrease in cleavage of an elastase-specific peptide substrate (succinyl-L-Ala-L-ALa-L-Ala-p-nitroanilide, Sigma-Aldrich) is a measure of PPE activity and is detected by colorimetric change. The α-test has been developed for serum (<0.5 ml test volume required) using the microtiter plate format (Bristow et al., 1998). The α-test has been determined to be valid only in HIV patients with >200 CD4 cells/μl. The linear relationship between α1PI and CD4+ lymphocytes allows use of a mathematical transformation to produce accurate CD4 T cell counts. Because whole saliva contains serum, the α-test can be used with either serum or saliva.

The α-test exploits the competition in complex fluids between α1PI and α2macroglobulin (α2M) for binding and inhibition of PPE. Both inhibitors bind PPE with 1:1 stoichiometry, but PPE will bind all the α2M before binding to α1PI because PPE has higher affinity for α2M than for α1PI. There is 10-fold less α2M than α1PI in blood so there is a dilution value at which α2M no longer has an effect, and PPE binds only to α1PI Residual PPE activity is measured by its cleavage of the elastase-specific peptide substrate. Thus, by diluting serum, it is possible to identify which inhibitor is acting to inhibit PPE, and this allows precise quantitation of the molar concentration of each inhibitor. Results are obtained by detecting a color change at 405 nm using a standard plate reader. Optical density units (OD405 nm) are transformed to molar concentration using a mathematical formula (Bristow et al., 1998). Mathematical transformation is performed using linear regression, and calculations are performed in any spreadsheet with mathematics capabilities such as Microsoft Excel.

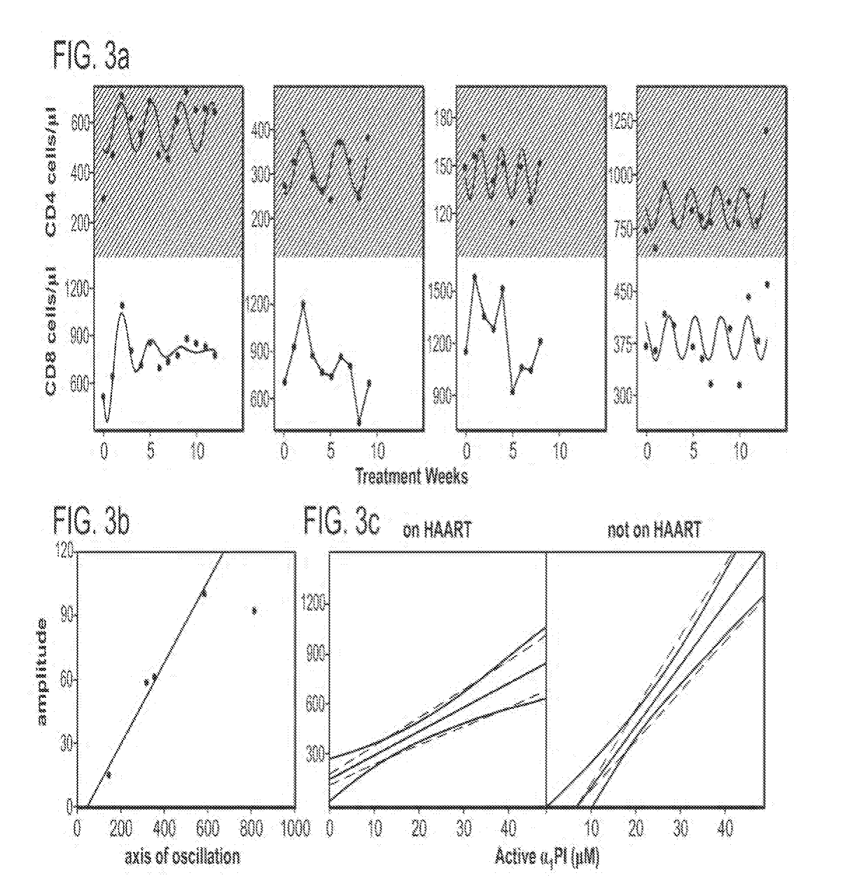

A goal for all CD4 enumeration methods is to monitor patients for sentinel events that signal a need to change medication. FIG. 3 shows that CD4 T cells cycle with periodicity 23 days (n=4). Circadian cycling in CD4 T cell counts has also been observed (Dimitrov et al., 2009). In addition to cycling, CD4 T cell counts change in response to immunization, infection, changes in antiretroviral medications, certain malignancies, stress, and immune failure. The cycling of CD4 T cells has not previously been taken into consideration in interpreting CD4 enumeration methods. The method for CD4 enumeration described herein incorporates CD4 cycling in establishing CD4 T cell reference ranges. Since CD4 T cells exhibit sinusoidal cycling, the axis of oscillation about which the CD4 T cells cycle allows for the first time, an accurate and precise measurement of CD4 T cell counts. The α-test measures active a α1PI in serum or saliva and calculates the CD4 T cell axis of oscillation.

Test Protocol

The protocol for performing the test according to preferred embodiments of the invention is as follows. Two-fold serial dilutions of serum or saliva are prepared in wells of a microtiter plate. To each well is added porcine pancreatic elastase (PPE, 0.2 U) for several min at room temperature after which is added a colorimetric elastase-specific peptide substrate (0.2 mM) for several min at room temperature. Color change is detected using a microtiter plate reader or a similar hand-held device. Regression analysis of the differences in color change in the serially diluted serum or saliva allows mathematically transformation of the data to the molar concentration of active α1PI in the patient sample as follows (Bristow et al., 1998): Log [Active α1PI (μM)]=−2 Log [PPE (Units)].

The linear relationship between α1PI and CD4 T cell count described herein allows calculation of CD4 T cells from the molar concentration of active α1PI using the equations described herein. This CD4 enumeration assay is called the α-test. The equations described herein are estimates of the true relationship between α1PI and CD4 T cell count. In further embodiments of the present invention it is envisioned that the equations may be modified as data accrue from future patient populations tested.

The α-test is reproducible because it incorporates internal controls that standardize the assay-to-assay and lab-to-lab variation. The primary source of variation in the assay is due to the addition of PPE to the sample wells. Variation in PPE added occurs due to variation in reagents, pipetting, time, temperature, and equipment. Standardization is accomplished by measuring the maximum PPE added to the diluted serum or saliva samples in each assay. Since PPE is added in excess, the PPE activity detected in the presence of serum us divided by the maximum PPE added, and this standardizes each assay to the maximum PPE added. Because each assay is standardized internally, the results are consistent, lab to lab, regardless of differences in water, PPE concentration, pipetting technique, substrate activity, or equipment. A simple linear transformation of the α1PI concentration yields CD4 T cell count. The assay is sensitive and specific for α1PI concentration (Bristow et al., 1998; Bristow et al., 2001), accurate and precise for CD4 enumeration.

The use of individual microtiter 8-well test strips further improves test performance and decreases the per test cost. These strips are individually read using a microtiter plate reader or a battery-operated, hand-held strip reader. The median values for α1PI in whole saliva and serum are 103 nM (Cox et al., 2006) and 33 μM (Bristow et al., 2001), respectively. It is possible to use saliva in the α-test even though whole saliva contains 300-fold less α1PI than serum. The software macro that converts strip-reader values to CD4 T cell count is straightforward due to the linear relationship between α1PI and CD4 T cell count.

Specimen Collection and Storage

1. Serum preparation: Blood is collected into tubes containing no additives and allowed to clot for 1½ hr. Serum is removed from the collection tube and can be used immediately.

2. Saliva collection: Saliva is collected by spitting into a cup or placing a sponge under the tongue. Saliva can be used immediately.

3. Specimen storage: Unused serum or saliva can be measured within 3 days of collection if stored at 4° C. or within one year if stored at −20° C.

Automation of the α-Test

The α-test uses a microtiter plate format and has been optimized for serum. This version of the α-test is accurate, precise, and cost efficient. The test also uses individual sample test strips and hand held detection devices to further decrease the per test cost. A software macro converts the raw data to values representing CD4 T cell counts.

Validation of the α-Test

The current version of the α-test performed in the microtiter plate format as described herein provides an accurate measure of CD4 T cell counts in HIV patients with >200 CD4 cells/ml. The statistical comparison of CD4 T cell counts from the α-test on serum and saliva was validated using the standard method that uses flow cytometry.

Longitudinal Monitoring of Patients

Patients are monitored for CD4 enumeration using the α-test with serum and saliva and these values are validated using standard flow cytometry CD4 enumeration in the context of HIV disease-related health events of each patient, e.g. immunizations, infections, and vacations from antiretroviral medications.

### Example 2

### Active α1PI is Rate Limiting for CD4+ Lymphocytes in HIV-1 Disease

In a first set of experiments, non-HIV-1 healthy volunteers were measured for active and inactive α1PI, and for the number of lymphocytes that were CD4+, CXCR4+, CCR5+, and HLECS+. Independently, neither active α1PI, HLECS+, CXCR4+, nor CCR5+ lymphocytes were correlated with CD4+ lymphocytes. However, by multilinear regression, it was found that higher CD4+ lymphocytes were correlated with a pair of counterbalancing variables, higher active α1PI (P=0.01) and lower HLECS+ lymphocytes (P=0.001) (r2=0.98, n=12, data not shown). In another study population, it was similarly found that CD4+ lymphocytes were correlated with the pair of counterbalancing variables active α1PI (<0.001) and blood cells expressing the stem cell marker CD34 (P=0.04) (r2=0.74, n=17, data not shown). However, in HIV-1 patients, this relationship does not hold. In early HIV-1 disease, active α1PI becomes abnormally low (Bristow et al., 2001), a situation that causes α1PI to be rate limiting for the migration of CD4+ lymphocytes from hematopoietic tissue into blood (Bristow et al., 2009).

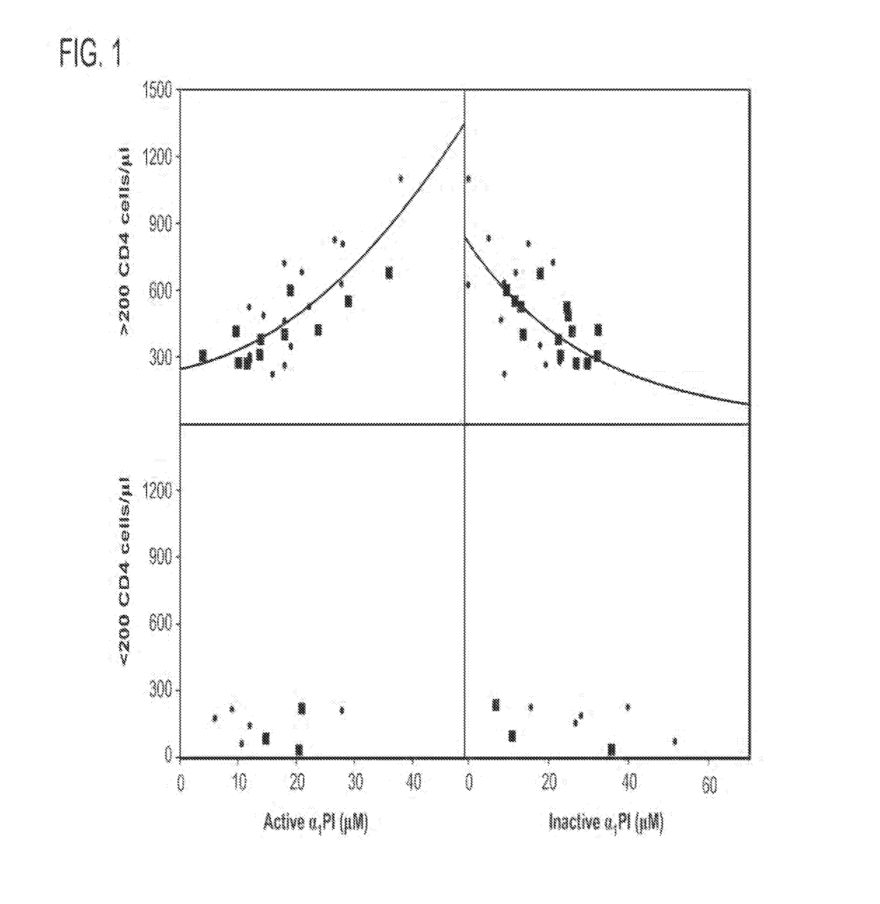

In 34 HIV-1 patients studied, 26 were above 200 and 8 were below 200 CD4 cells/μl at the time of blood collection. All patients were measured for HIV RNA copies/ml, CD4, CXCR4, CCR5, CXCL12 levels, active and inactive α1PI. In the 26 patients with >200 CD4 cells/μl, higher CD4+ lymphocyte levels were correlated with higher active α1PI concentration (r^{2=0.94}) and lower inactive α1PI concentration (r^{2=0.95}) (FIG. 1). In patients with <200 CD4 cells/μl, there was no relationship between CD4+ lymphocyte levels and active or inactive α1PI, and this suggests either HIV-1 itself, or other host processes had contributed to disrupting the regulation of CD4+ lymphocyte levels. As in the non-HIV-1 population, neither CXCR4 nor CCR5 were found to correlate individually or in combination with any parameters of disease being investigated in these patients.

### Example 3

### Predicting CD4 T Cell Counts

Predicting CD4 T cell counts from active α1PI is optimized when patients are partitioned into two groups, those patients receiving HIV protease inhibitor therapy (PI) and those who are not.

The computational method for CD4 enumeration is due to the linear relationship between active α1PI and CD4 T cell counts. The well-known effect of HIV protease inhibitor therapy to increase CD4 T cell counts may in part result from effects that simulate the physiologically relevant α1PI effect.

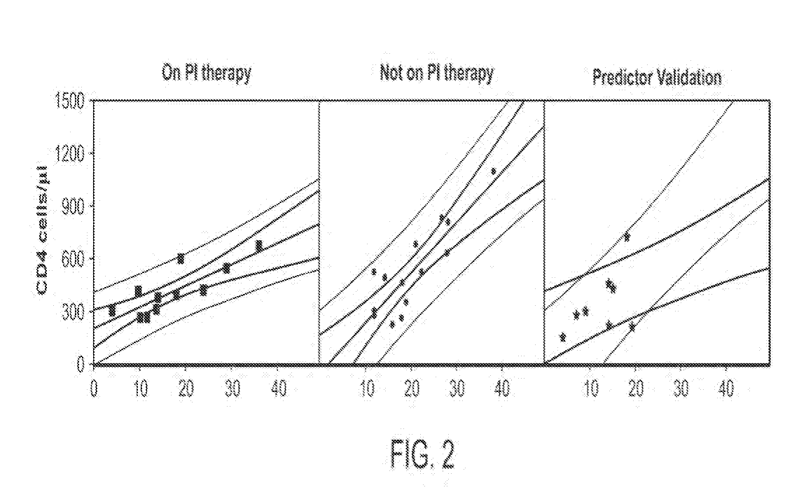

Patients with >200 CD4 cells/μl depicted in FIG. 1 were partitioned into two groups with respect to HIV PI therapy (FIG. 2). Regression analysis with confidence limits and prediction limits are depicted. Using active α1PI to predict CD4 T cell count was validated using an additional 8 patients which entered the study subsequent to the patients depicted in FIG. 1. All new patients fell within the prediction limits thereby validating this CD4 enumeration methodology.

### Example 4

### α1PI Augmentation Therapy Increases CD4 T Cell Counts in HIV and Non-HIV Patients

CD4 T cell counts are driven by the concentration of α1PI, and this is the reason that measuring α1PI provides an ideal cell-free surrogate for measuring the number of CD4+ T cells in blood.

The interrelationship between α1PI concentration and CD4+ lymphocyte numbers suggested the possibility that α1PI might regulate the number of CD4+ lymphocytes. Data were examined from a blinded study conducted by CSL Behring to monitor hematologic changes following weekly infusions of α1PI (ZEMAIRA, 60 mg/kg) in 11 individuals with genetic α1PI deficiency (PI_{ZZ}) who had never before received α1PI therapy. Treatment with α1PI was found to cause an increase in lymphocytes in 10 of these individuals (data not shown), and this suggested that α1PI regulates CD4+ lymphocyte numbers.

To determine whether α1PI therapy might benefit HIV-1 patients by inducing increased CD4+ lymphocyte numbers, a pilot study was conducted involving 3 HIV-1 patients at different stages of disease and 2 patients with genetically determined α1PI deficiency (PI_{ZZ}) (Bristow et al., 2009). Because chemokine-related circadian variation in absolute CD4 T cell counts have been reported (Dimitrov et al., 2009), HIV-1 patients Alpha, Beta, and Gamma and both PI_{ZZ} patients each received weekly treatment at the same time of day of the same day of the week thus eliminating circadian variation. To control for assay-related variation, CD4 T cell count was determined in two separate labs.

All patients responded to therapy with an initial burst of lymphocytes. After 2 wks of therapy, patient Alpha and Beta achieved normal numbers of CD4+ lymphocytes with increases from 297 to 710 and from 276 to 393 cells/α1, respectively. Patient Beta had never been in the normal range of CD4 T cell counts in more than 20 yrs. Patient Gamma, whose disease had progressed to the stage of immune failure, demonstrated a weak response to α1PI therapy. Patients PI_{ZZ}-1 and PI_{ZZ}-2 increased from 743 to 954 and from 899 to 1024 cells/α1, respectively. In parallel, CD8+ lymphocytes decreased, but there was no effect on other blood cell numbers. A conclusion from these results if that α1PI regulated the number of CD4+ lymphocytes in HIV-1 patients.

### Example 5

### CD4+ Lymphocytes Exhibit Sinusoidal Cycling

The standard methods for monitoring CD4 T cell counts have not previously taken into account the 23-day cyclic changes. The results described herein show that cycling occurs, thus it is necessary to consider cycling in determining the accuracy and precision of CD4 enumeration assays.

Thymopoiesis in humans has not previously been characterized, but in adult mice thymopoiesis is a multi-step process that is highlighted by a 21 day cycle of coordinated journeying of progenitor cells between adult bone marrow and thymus (Donskoy et al., 2003). Patients Alpha, Beta, and PI_{ZZ} exhibited sinusoidal changes in CD4+ lymphocytes with periodicity 23±3.5 days (Bristow et al., 2009). Cyclic changes in Patient Gamma exhibited a periodicity of 15 days suggesting the presence of additional disturbances in the regulation of CD4 T cells. Additional investigations are currently underway to determine whether the influence of α1PI on CD4+ lymphocyte numbers is due to participation in adult thymopoiesis.

FIG. 2 shows the 95% confidence limits and prediction limits for the α-test. A question arises as to how much of the variation in predicting CD4 T cell counts is due to CD4 cycling and how much is due to the limitation of accuracy in the test.

Sine curve analysis of the CD4+ lymphocyte changes in the 5 patients in the study (FIG. 3a) yielded values for peak-to-peak amplitude and axis of oscillation that were unique to each patient (Bristow et al., 2009). In patients with low CD4 T cell counts, the cyclic changes in CD4 T cell count were small, and in patients with high CD4 T cell counts, cyclic chart were large. In practical terms, this means that CD4 T cell counts vary by as much as 200 cells/μl between the nadir and apex with a periodicity of 23 days, and this is true regardless of the method for measuring CD4 T cell count. Therefore, the axis of oscillation provides a more accurate measure of the true CD4 T cell count since this value is constant hour to hour and week to week.

As expected, amplitude was correlated with axis of oscillation, and this relationship was linear (r2=0.999), reaching a plateau at an amplitude value of 100 CD4 cells/μl (FIG. 3b). This means that if a patient has high CD4 T cells counts, the axis of oscillation and amplitude are both high, and vice versa, if a patient has low CD4 T cell counts, the axis of oscillation and amplitude are both low.

Because the relationship between α1PI and CD4 T cell count is linear and because there is a sufficiently high correlation, it can be inferred that the more data points used to estimate the regression parameters, the closer the regression lines in FIG. 2 will come to represent, not simply CD4 T cell count, but the actual axis of oscillation for each patient. In other words, CD4 T cell counts that do not fall exactly on the regression lines in FIG. 2, do so, in part, because of cycling. How far above and below the regression line the CD4 T cell counts will vary due to cycling can be calculated because the axis of oscillation (regression line) is correlated with amplitude (FIG. 3b). When the expected amplitude for each point on the regression line was calculated, it was found that the amplitude overlapped the 95% the confidence intervals (FIG. 3c). When the amplitude and confidence intervals for the regression lines were compared by Mann-Whitney Rank Sum Test, it was found that there was no significant difference. The identity between the expected amplitude and confidence intervals is interpreted to mean that there is 95% confidence that the reason the actual CD4 T cell counts do not fall exactly onto the regression lines in FIG. 2 is due to chiefly to CD4 cycling.

Accuracy is a measure of the similarity between CD4 T cell counts using new technology versus standard technology (flow cytometry). Based on the analysis above, the α-test is accurate with 95% confidence in comparison with standard flow cytometry that was performed at an independent lab.

Precision is a measure of reproducibility using a single technology, and this can be represented by coefficient of variation (CV, ratio of standard deviation to the mean). By measuring mean and standard deviation of CV % in 4 repeats of 3 patients, the precision of the α-test was found to be 3.5%+/−2.5%.

Sensitivity is a measure of how well a test identifies a positive result that is true positive, e.g., patients who have critical CD4 T cell counts. Specificity is a measure of how well a test identifies a negative result that is a true negative. The sensitivity and specificity of the α-test as depicted in Table 1 has been previously determined (Bristow et al., 2001):

Table 1, shown below, shows indicators for transition between clinical categories in HIV disease.

TABLE 1

| | CDC | WHO | Sensitivity | Specificity |
|---|---|---|---|---|
| Test | Staging ² | Staging ³ | (%) ¹ | (%)* |
| α-test | A1, A2 to B1, B2 | I to II | 77.8 | 100 |
| | B1, B2 to AIDS | II to IV | 100 | 80.6 |
| CD4 (flow | A1, A2 to B1, B2 | I to II | 83.3 | 92.9 |
| cytometry) | B1, B2 to AIDS | II to IV | 100 | 97.2 |
| HIV RNA | A1, A2 to B1, B2 | I to II | 66.7 | 78.6 |
| | B1, B2 to AIDS | II to IV | 85.7 | 69.4 |

In Table 1,
¹ Sensitivities and specificities were determined from data represented in FIG. 1 using CART analysis (classification and regression trees) by the Biostatistics Core of the UNC Center for AIDS Research.
² Clinical category of disease as defined by the CDC 1993 revised classification (Castro et al., 1992).
³ Clinical category of disease as defined by the WHO 2005 updated classification (WHO, 2005).

The conclusion from the above analysis is that the α-test is accurate and precise in HIV patients with undetectable viral load. The α-test is as sensitive as flow cytometry in identifying disease category except in AIDS, and this difference is due to the increase in viral load that occurs during AIDS.

### Example 6

### CD4+ Lymphocytes Increase and CD8+ Lymphocytes Decrease in Response to α1PI Replacement Therapy

In earlier work, it was established that antibodies reactive with HIV-1 gp120 also bind and inactivate human α1PI, producing IgG-α1PI immune complexes (6). IgG-α1PI immune complexes produce functional α1PI deficiency in HIV-1 infected individuals. A single amino acid differentiates chimpanzee α1PI from human α1PI, and this difference is in the HIV-1 gp120 homologous domain, perhaps explaining the lack of progression of HIV-1 infected chimpanzees to AIDS (7). Further, comparison of the amino acid sequences of human α1PI, HIV-1, HIV-2, SIV, HTLV-1, and HTLV-2 reveals that all share homology with the hydrophobic core of the fusion domain of HIV-1 gp41 (LFLGFL), but only HIV-1 gp120 shares homology with α1PI (6). It was then hypothesized that the insufficient α1PI that attends HIV-1 disease might secondarily cause CD4+ lymphocytes to become trapped in tissue, unable to complete the second step of cell migration and be released into blood. The experiments described herein demonstrate that α1PI augmentation induces substantial increases in lymphocytes that cycle with a 23±3.5 day periodicity.

To examine the interrelationship between α1PI concentration and CD4+ lymphocyte numbers, data were examined from a blinded study conducted to monitor hematologic changes following weekly infusions of α1PI (60 mg/kg) in 11 individuals with genetic α1PI deficiency (PI_{ZZ}) who had never before received α1PI therapy. Treatment with α1PI caused an increase in lymphocytes in 10 of these individuals (data not shown), suggesting that such treatment might benefit HIV-1+ patients.

Two HIV-1 patients, Alpha and Beta at different stages of disease (Table 1) received weekly infusions of 120 mg/kg α1PI augmentation. Table 2 that shows the HIV-1 population at baseline, is shown below.

TABLE 2

| | | | | | CD4³ | HIV |
|---|---|---|---|---|---|---|
| | NRT/ | | HIV-1⁺ | α₁PI³ | cells/ | RNA³ |
| Patient ¹ | NNRT/PI | Age | since | (μM) | μl | copies/ml |
| Alpha | Epivir/Sustiva/ | 47 | 2001 | 9 | 297 | <400 |
| | none | | | | | |
| Beta | Combivir/ | 53 | 1982 | 7 | 276 | <400 |
| | Sustiva/none | | | | | |
| Gamma | Combivir/ | 70 | Unknown² | 4 | 148 | <400 |
| | Viramune/Kaletra | | | | | |
| Delta | Truvada/ | 51 | 1982 | 14 | 445 | 205 |
| | Sustiva/none | | | | | |

¹ All patients were at different stages of HIV-1 disease progression and were on antiretroviral medication with adequate suppression of virus.
²Infected for many years, and first tested Jan. 3, 2005.
³Serum levels.

Two additional HIV-1 patients, Gamma and Delta, with a priori evidence of abnormal immune status received the same therapy. The ability of Gamma to respond to antigen was impaired (positive PPD followed by negative PPB), and Delta exhibited systemic inflammation. Finally, 2 non-HIV-1 patients were included in the study, PI_{ZZ}-1 and PI_{ZZ}-2, who manifested normal numbers of CD4+ lymphocytes and a diagnosis of emphysema in the context of genetic α1PI deficiency. The PI_{ZZ} patients received half-dose weekly infusions of 60 mg/kg α1PI augmentation. Patients Delta and PI_{ZZ}-2 were included only in CD4+ lymphocyte functional analyses (see Methods).

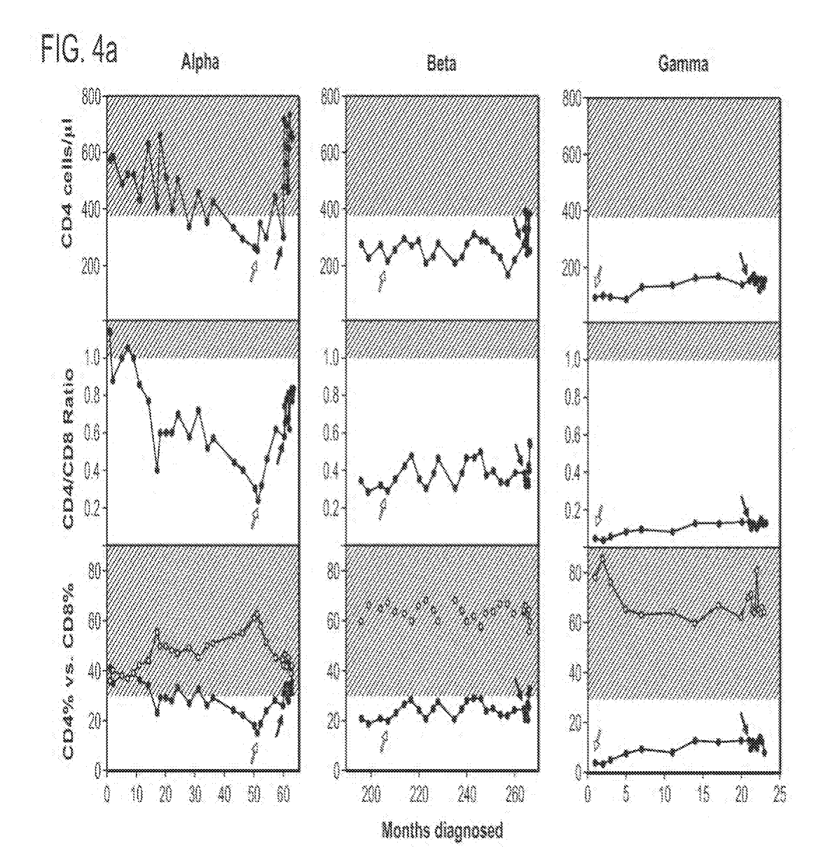

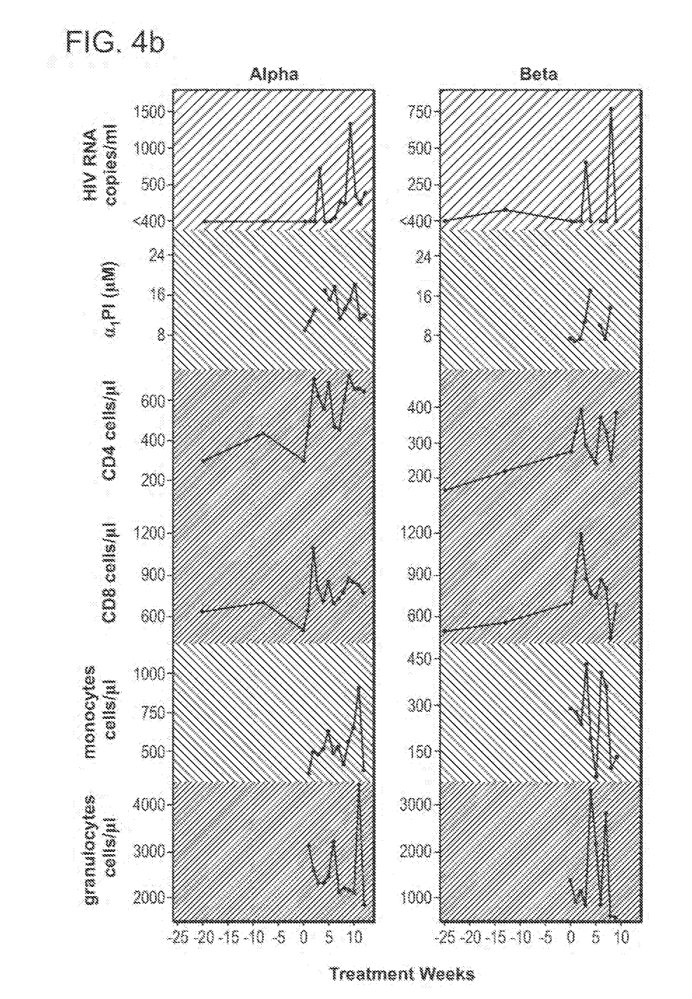

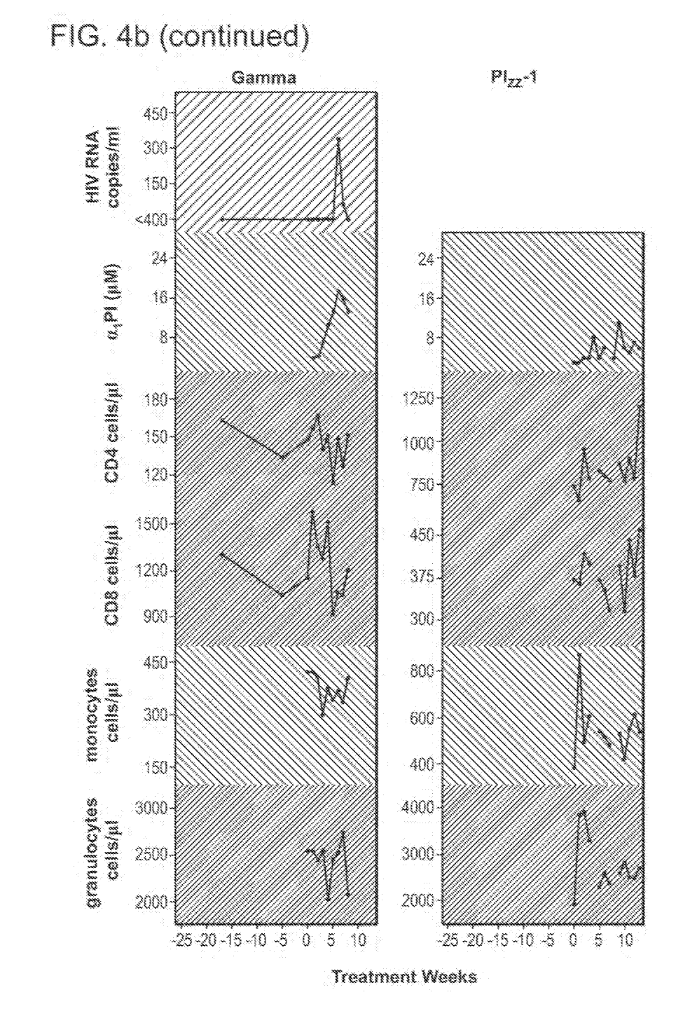

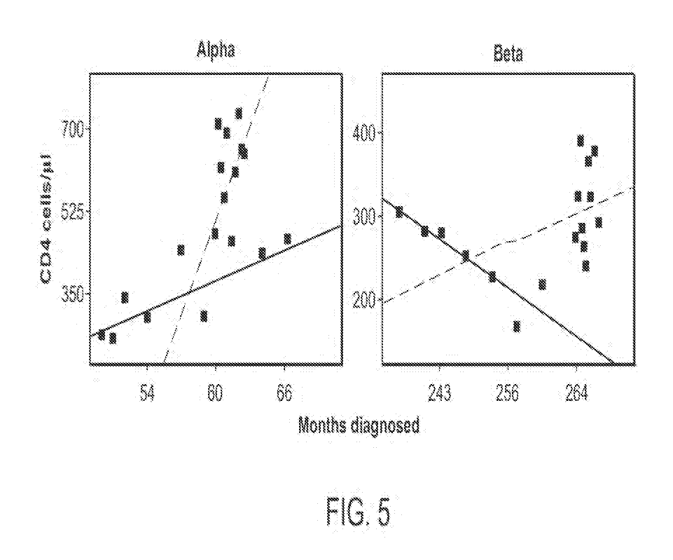

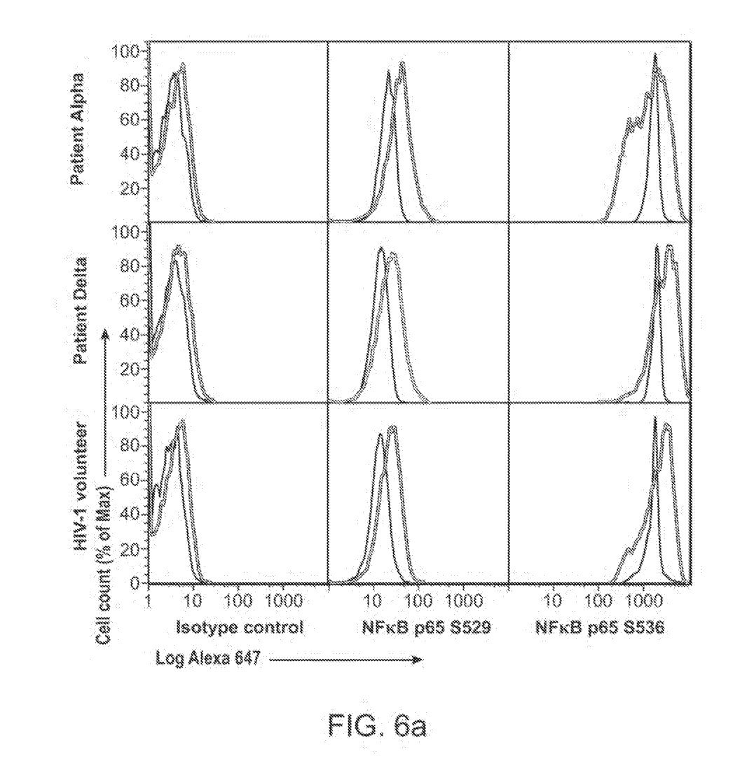

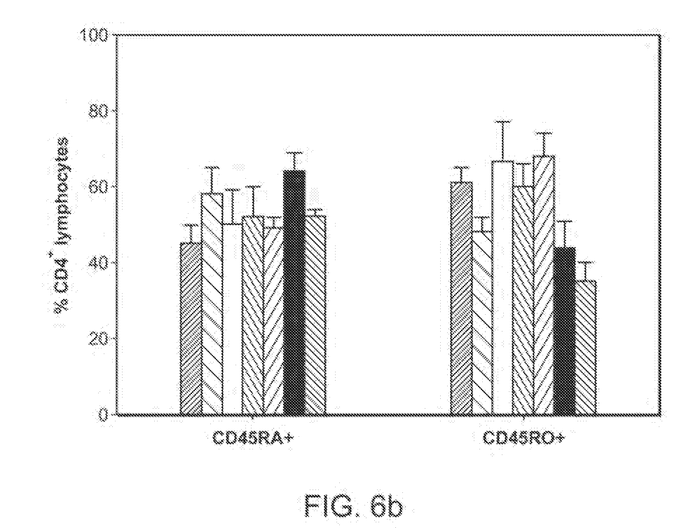

HIV-1+ patients Alpha, Beta, and Gamma (FIG. 4) and both PI_{ZZ} patients (not depicted) responded to therapy with an initial burst of lymphocytes. After 2 wks of therapy, patients Alpha and Beta achieved normal numbers of CD4+ lymphocytes with increases from 297 and 710 and from 276 to 393 cells/pl, respectively. Patients PI_{ZZ}-1 and PI_{ZZ}-2 increased from 743 to 954 and from 899 to 1024 cells/pl, respectively. Patient Beta, who had never exhibited CD4+ lymphocytes in the normal range in more than 20 years, even continued to exhibit the normal range of CD4+ lymphocytes 2 wks after treatment stopped with 382 cells/pl. Using regression analysis, this duration of benefit was attributed to α1PI therapy (FIG. 5). Patient Alpha who was first infected 5 years prior to the study had not exhibited CD4+ lymphocytes within the normal range in 2 years. At 5 wks and 14 wks after treatment stopped, patient Alpha continued to be in the normal range with 470 cells/pl. By regression analysis, this duration of benefit appeared to be related to antiretroviral medication as well as α1PI therapy (FIG. 5). Patient Gamma, who was known to have lost immune function, showed an increase from 148 to 167 cells/pl and never achieved normal numbers of CD4+ lymphocytes.

### Example 7

### Expanded CD4+ Lymphocytes are Phenotypically Mature and Respond to Stimulation

Glucocorticoids cause the release of granulocytes and lymphocytes from tissue, but the peak increase occurs within 6 hrs and dissipates within 24 hrs (8). Glucocorticoid-induced demarginalized lymphocytes are unresponsive to stimulation. To determine whether α1PI-mobilized CD4+ lymphocyte populations were functional, CD4+ lymphocytes were isolated from blood and cultured in stimulation media. Harvested culture supernatants were quantitated for a panel of cytokines representing subpopulations of CD4+ lymphocytes, specifically IL-2 (Th1), IL-4, (Th2+NKT), IL-10 (Th2), and IFNγ (Th1+NKT). Harvested cells were examined for NFκB activation. Whether isolated fro HIV-1 uninfected volunteers or from HIV-1 patients pre-treatment or under treatment, CD4+ lymphocyte populations were equivalently capable of being stimulated (Table 2, FIG. 7). Table 2, shown below, shows stimulated cytokine release. In Table 2, cytokines were measured in culture supernatants harvested from isolated CD4+ T cells stimulated by antibodies reactive with CD2, CD3, and CD28. Harvested CD4+ T cells were simultaneously monitored for NFêB activation (FIG. 7). In two attempts, the number of CD4+ lymphocytes isolated from Patient Gamma was insufficient for stimulation analysis. Cytokines were undetectable in serum or culture supernatants from unstimulated CD4+ T cells with the exception of Patient Delta who exhibited 51 pg IL-2/ml in his baseline serum sample, and Patient Beta who exhibited 14 pg IL-10/ml in serum as treatment week 8. In Table 3, n/d stands for not determined.

TABLE 3

| | Treatment | IL-2 | IL-4 | IL-10 | INFγ |
|---|---|---|---|---|---|
| Patient | Week | (pg/ml) | (pg/ml) | (pg/ml) | (pg/ml) |
| Alpha | 11 | >800 | <31.2 | 148 | >500 |
| Beta | 6 | 792 | <31.2 | <7.8 | >500 |
| Delta | untreated | >800 | <31.2 | n.d.² | >500 |
| non- | untreated | >800 | <31.2 | 239 | >500 |
| HIV-1 | | | | | |

CD4+ lymphocytes in whole blood were analyzed by FACS analysis for phenotypic markers characteristic of mature and immature, activated and quiescent cells including CD34 (stem cells), CD8 (double positive and double negative immature cells), CD45RA (naïve cells): CD45RO (memory cells). CD25 (IL-2Ra activated cells and thymocytes), CXCR4 and CCR5 (HIV-1 tropism-determining chemokine receptors). Patients Alpha and PIzz-1 expressed significantly greater CD4CD45RA+ naïve cells than did the HIV-1 uninfected volunteers (FIG. 7b). In parallel, patient Alpha and PIzz-1 as well as PIZZ-2 expressed significantly lower CD4+CD45RO+ memory cells than the HIV-1 uninfected volunteers. However, these differences did not appear to be related to α1PI therapy since the percentage of naïve and memory cells remained steady in each patient throughout therapy. All other surface markers measured were normal (not depicted). These results suggest that the phenotypic profile of CD4+ lymphocytes unique to each individual was maintained within the new generation of lymphocytes.

The processes that renew the adult human CD4+ lymphocyte population are poorly understood and the sinusoidal changes we observed may offer insight into the renewal mechanisms. While cyclic variation in circulating CD4+ lymphocytes has not been previously described, cyclic neutropenia occurs with and average 21-28 day periodicity and is caused by mutations in the α1PI receptor, HLE_{CS} (9). The α1PI-induced in situ proliferation of CD4+ lymphocytes (24-48 h) (10) or the release of functional CD4+ lymphocytes from lymph tissue into circulation as occurs in an acute phase reaction (4-24 h) (11) may explain cycling, but neither of these explanations account for the 2 wk lag in appearance and 23 day periodicity. Rather, a multi-step process is implied.

In adult mice, thymopoiesis is a multi-step process defined by a 21-day cycle. The process involves the cyclic accumulation of progenitor cells in the adult bone marrow (3-5 wk), export of progenitor cells from the bone marrow thereby vacating the bone marrow niche (˜1 wk), temporally coordinated importation of progenitor cells into blood, and repopulation of the bone marrow niche at the end of the cycle (12). When positive selection of CD4+ thymocytes is impaired, the number of CD4+ lymphocytes diminishes by up to 80% (13), and as a consequence the number of CD8+ lymphocytes increases thereby producing a decreased CD4/CD8 ratio (13,14).

In the analogous human system, HLECS, CXCR4, and CXCL12 are required for progenitor cells to vacate bone marrow, (2,15). In the present study, a lower pre-treatment CD8 percentage was inversely correlated with α1PI-induced expansion of CD4+ lymphocytes as would be expected if treatment had re-established positive selection. Thus, we propose that the observed α1PI-induced changed in CD4+ and CD8+ lymphocyte numbers resulted from the binding of α1PI to the CXCR4/HLECS/CXCL12 complex during cell migration thereby facilitating adult thymopoiesis. In contrast to all other patients in the study who exhibited a 3-week cycle, patient Gamma exhibited a 2-week cycle suggesting some parts of the thymopoiesis cycle were intact, but that at least one step was impaired. This patient was documented to have lost the ability to respond to PPD which is a T lymphocyte-mediated response and is clinically interpreted to mean loss of immune function. In support of this hypothesis, there was no evidence of increased numbers of T lymphocytes in this patient suggesting that progenitor cells were being released from bone marrow, but not establishing residence in the stem cell niche of the thymus.

The phenomenon that PI_{ZZ} patients exhibit deficient hepatocyte-synthesized α1PI from birth, yet manifest normal numbers of CD4+ lymphocytes suggests there are additional considerations during fetal thymic selection, possibly the thymic or stromal supply of α1PI. Both myeloid and lymphoid cells are known to synthesize α1PI in bone marrow (16,17) Alternatively, the role of α1PI in hematopoiesis might be supplanted by other proteinase inhibitors during fetal development.

The duration of benefit for 2 wks, but not 5 wks, post-treatment suggests α1PI augmentation might be effective with less frequent than weekly administration. Our results predict that α1PI augmentation may overcome a localized pathologic system thereby allowing the immune system to recover and regain production of normal numbers of CD4+ lymphocytes in a subset of HIV-1+ patients who are on antiretroviral therapy and have functioning lymphatic and hematopoietic tissue.

### Example 8

### Calculations of CD4 T-Cell Counts

CD⁴+ T cell numbers are calculated by linear transformation of α₁PI concentration. CD4 T cell count in patients on PI therapy are calculated using, for example, the equation shown below:

CD4 cells/μl=205+12α1PIμM.

CD4 T cell count in patients not on PI therapy will be calculated using, for example, the equation shown below:

CD4 cells/μl=−60+29α1PIμM.

The two equations shown above are estimated based on 15 and 11 patients, respectively, and can be easily modified using a larger number of patients to more accurately reflect the true relationships

CD4⁺ T cell numbers are also calculated using, for example, the equation shown below:

CD4+ T cells/μl=b(0)+b(1)×α1PIμM (Formula III)

wherein α1PI is calculated as α₁PI activity (OD₄₀₅ₙₘ).

For a patient receiving PI therapy b(0) is between 75 and 385 and b(1) is between 6 and 18.

For a patient not receiving PI therapy b(0) is between −200 and 50 and b(1) is between 25 and 35.

CD4⁺ T cell numbers are also calculated, for example in saliva, using, for example, the equation shown below:

Log [CD4 (cells/μl)]=b(0)+b(1)×Log [α₁PI Index] (Formula IV)

wherein α₁PI Index is calculated as α₁PI activity (OD₄₀₅ₙₘ)/serum concentration in saliva (OD₅₉₅ₙₘ), b(0) is between 2 and 3 and b(1) is between 0.2 and 0.3.

Quantitation of α₁PI activity is determined by measuring inhibition of the elastase-mediated cleavage of a colormetric substrate. Color change is determined by measuring light scatter at the color specific wavelength and is measured in units of optical density (OD). OD units are plotted against the dilution factor for the complex mixture being measured, e.g. serum or saliva. In serum and saliva, α₁PI is in competition with another elastase inhibitor, α₂Macroglobulin (α₂M). Due to differences between in elastase inhibition and concentration in serum and saliva, the plot between OD and dilution factor yields a “V” shaped curve. The nadir of the “V” is represents the dilution where α₁PI and α₂M are equal in concentration. The OD at the nadir of the “V” is concerted to α₁PI molar concentration using the equation:

Log [OD]=(−2) log [dilution factor].

The calculations described above that are needed to convert OD units to α₁PI molar concentration and CD4 T cell counts can be performed manually using a calculator or can be performed in a spreadsheet with mathematical capabilities. To facilitate the delivery of CD4 T cell counts, a pre-programmed macro that performs the described calculations will be embedded in a spreadsheet with mathematical capabilities. Alternatively, the delivery of CD4 T cell counts is by presentation in a table representing all possible outcomes.

### MATERIALS

The invention and results described herein are performed with, but not limited to, the following materials and methods.

### Human Subjects

It was determined using the empirical correlation between α1PI and CD4+ lymphocytes that a sample size at 2 HIV-1+ patients would be adequate two achieve a significance level with alpha=0.05 and power of test=0.8 between pre- and post-treatment CD4+ lymphocytes levels. Inclusion criteria for treatment were: i) active α1PI below 11 μM; ii) one year history with CD4+ lymphocytes at levels ranging between 150 and 300 cells/μl; iii) absence of symptoms suggestive of HIV-1 disease progression; iv) adequate suppression of virus (<50 HIV RNA/ml); and v) history of compliance with antiretroviral medication. Due to the small size of the study and to avoid other complications of pregnancy, only male HIV-1 patients were enrolled. The half-life of ZEMAIRA (purified α1PI) after infusion is 4.5 days, reaching steady state after 3-4 wks therapy (18). CSL Behring contributed a sufficient quantity of ZEMAIRA (lot# C405702) for administration of 8 weekly infusions at a dose of 120 mg/kg.

Written informed consent was received from 4 HIV-1 patients designated Alpha, Beta, Gamma, and Delta. For comparison, blood was collected from 2 non-HIV-1 patients, both female, with a diagnosis of emphysema in the context of genetic α1PI deficiency (PI_{ZZ}-1 and PI_{ZZ}-2, ages 52 and 53, respectively). In all cases, patients had never before received α1PI augmentation therapy. After initiating therapy, an assessment of dosage and the quantity of donated ZEMAIRA allowed for the extended treatment of patient Alpha for a total of 12 wks.

Due to an insufficient number of serum samples from patient PI_{ZZ}-2, only functional analyses of CD4+ lymphocytes are presented. Patient Gamma who was PPD positive and elderly had become PPD negative 2 yrs prior to the treatment presented here which is clinically interpreted as a loss of immune function. Patient Delta reported to the first infusion stating than due to unforeseen circumstances, his antiretroviral medication was interrupted for 4 days. Although there was no fever present or other indication of infection at the time of the first infusion, in follow-up analysis, this patient was found to have pre-treatment serum IL-2 levels of 51 pg/ml (normal is undetectable) and other atypical baseline measures indicative of an inflammatory response and exceeding study inclusion criteria including 454 CD4+ cells/μl, 205 HIV RNA copies/ml, and 14 μM α1PI. Thus, blood from patient Delta was analyzed for the purpose of assessing treatment response in the presence of systemic inflammation. Only pre- and post-treatment NFκB activation, cytokine release, and lymphocyte phenotype were determined for this patient.

Blood was collected at each session and was seat to a contractor medical laboratory which provided independent measurement of the complete blood cell count (CBC) with differential, lipid panel, blood chemistry, lymphocyte panel, and HIV RNA. Periodically, kidney (BUN and creatinine) and liver function tests (ALT, AST) were monitored for potential immune complex disease, and all measurements were within the normal range. Lymphocyte function and phenotype analysis were performed by our laboratory. The study protocol was approved by CSL Behring and by the institutional review board of Cabrini Medical Center. No adverse effects were reported by any patient.

### Serum α1PI Levels

Active α1PI was determined in once-thawed serum samples by our laboratory as previously described with the modification that end-point, rather than kinetic, analysis was measured (19). Briefly, the inhibition of porcine pancreatic elastase (PPE, Sigma, St. Louis, Mo.) that was specifically attributable to serum α1PI was quantitated in the context of the serum concentration of α2microglobulin and its higher affinity for PPE relative to α1PI.

### Lymphocyte Phenotype Analysis

Surface staining on whole blood was performed by incubating for 15 min at 23° C. with ASR type, fluorescently conjugated antibodies recognizing CD4, CD3, CD8, CD45RA, CD45RO, CXCR4, CCR5, CD34, CD25, and isotype controls (BD Biosciences). Cells wore subsequently stained to detect HLECS by incubating whole blood for an additional 15 min at 23° C. with rabbit anti-HLE (Biodesign, Kennebunkport, Me.) or negative control rabbit IgG (Chemicon, Temecula, Calif.) which had been conjugated to Alexa Fluor 647 (Molecular Probes). At least 10,000 cells front each sample were acquired using a FACSCalibur flow cytometer. Markers on cells in the lymphocyte gate were quantitated, and CD4+ cells in the lymphocyte gate were validated using a contractor medical laboratory. Cell staining was analyzed using CellQuest (BD Biosciences) or FlowJo software (Tree Star, Inc., Ashland, Oreg.).

### CD4+ Lymphocyte Functional Analysis

CD4+ lymphocytes were negatively selected from PBMC using magnetic cell sorting as recommended by the manufacturer (Miltenyl Biotec, Auburn, Calif.). Isolated cells (1×10⁶ cells/ml) were cultured in medium containing 10% FBS in 24-well tissue culture plates for 3 days at 37° C., 5% CO2, in the presence or absence of stimulation antibodies reactive with CD2, CD3, and CD28 as recommended by the manufacturer (Miltenyl Biotec). Culture supernatants were measured by ELISA as recommended by the manufacturer (R&D Systems, Minneapolis, Minn.) for IL-2, IL-4, IL-10, and IFNγ.

Harvested CD4+ lymphocytes were examined for NFKB phospho-epitope staining by flow cytometry as previously described (20). Briefly, 1×10⁶ cells/well in 96 well plates were feed using 1.5% paraformaldehyde, washed with PBS containing 1% BSA, and incubated at 4° C. for 10 min in 100 μl ice-cold methanol. Cells were washed and incubated at 23° C. for 20 min with phosphoprotein-specific antibodies (BD Pharmingen and BP Phosflow, San Diego, Calif.) directly conjugated with Alexa Fluor 647 (Molecular Probes Invitrogen, Carlsbad, Calif.).

All publications and patent documents cited in this application are incorporated by reference in their entirety for all purposes to the same extern as if each individual publication or patent document were so individually denoted. By their citation of various references in this document, Applicants do not admit any particular reference is “prior art” to their invention. The following specific references, also incorporated by reference, are indicated above by corresponding reference number.
1. Cepinskas, G., M. Sandig, and P. R. Kvietys. 1999. PAF-induced elastase-dependent neutrophil transendothelial migration is associated with the mobilization of elastase to the neutrophil surface and localization to the migrating front. J. Cell Science 112: 1937-1945.
2. Lapidot, T. and I. Petit. 2002. Current understanding of stem cell mobilization: The roles of chemokines, proteolytic enzymes, adhesion molecules, cytokines, and stromal cells. Exp. Hernatol. 30:973:-981.
3. Bristow, C. L., D. R. Mercatante, and R. Kole. 2003. HIV-1 preferentially binds receptors co-patched with cell surface elastase. Blood 102;4479-4486.
4. Cao, C., D. A. Lawrence, Y. Li, C. A. Von Amin, J. Herz, E. J. Su, A. Makarova, B. T. Hyman, D. K. Strickland, and L. Zhang. 2006. Endocytic receptor LRP together with tPA and PAI-1 coordinates Mac-1-dependent macrophage migration. EMBO J. 25:1860-1870.
5. Kounnas, M. Z., F. C. Church, W. S. Argraves, and D. K. Strickland. 1996. Cellular, internalization and degradation of antithrombin III-thrombin, heparin cofactor II-thrombin, and alpha 1-antitrypsin-trypsin complexes is mediated by the low density lipoprotein receptor-related protein. J. Biol. Chem. 271:6523-6529.
6. Bristow, C. L., H. Patel, and R. R. Arnold, 2001. Self antigen prognostic for human immunodeficiency virus disease progression. Clin Diagn. Lab. Immunol. 8:937-942.
7. Huber, R. and R. W. Carrell. 1989. Implications of the three-dimensional structure of alpha 1-antitrypsin for structure and function of serpins. Biochemistry 28:8951-8966.
8. Chrousos, G. P. 2007. Adrenocorticoids & Adrenocortical Antagonists. In Basic & Clinical Pharmacology, 10th Edition. B. G. Katzung, ed. McGraw-Hill Companies.
9. Horwitz, M., K. F. Benson. R. E. Person. A. G. Aprikyan, and D. C. Dale. 1999. Mutations in ELA2, encoding neutrophil elastase, define a 21-day clock in cyclic haematopoiesis. Nat. Genet. 23:433436.
10. Congote, L. F. and N. Temmel. 2004. The C-terminal 26-residue peptide of serpin A1 stimulates proliferation of breast and liver cancer cells; role of protein kinase C and CD47. FEBS Lett. 576:343-347.
11. Mehigan, B. J., J. E. Hartley, P. J. Drew, A. Saleh, P. C. Dore, P. W. Lee, and J. R. T. Monson. 2001. Changes in T cell subsets, interleukin-6 and C-reactive protein after laparoscopic and open colorectal resection for malignancy. Surg. Endosc. 15:1289-1293.
12. Donskoy, E., D. Foss, and I. Goldschneider. 2003 Gated importation of prothymocytes by adult mouse thymus is coordinated with their periodic mobilization from bone marrow. J. Immunol. 171:3568-3573.
13. Nakagawa, T., W. Roth, P. Wong, A. Nelson, A. Farr, J. Deussing, J. A. Villadangos, H. Ploegh, C. Peters, and A. Y. Rudensky. 1998. Cathepsin L: Critical Role in Ii Degradation and CD4 T Cell Selection in the Thymus. Science 280:450-453.
14. He, X. and D. J. Kappes. 2006. CD4/CD8 lineage commitment:light at the end of the tunnel? Curr. Opin. Immunol. 18:135-142.
15. Tavor, S., I. Petit, S. Porozov, P. Goichberg, A. Avigdor, S. Sagiv, A. Nagler, E. Naparstek, and T. Lapidot. 2005. Motility, proliferation, and egress to the circulation of human AML cells are elastase dependent in NOD/SCID chimeric mice. Blood 106:2120-227.
16. Bashir, M. S., K. Morrison., D. H. Wright, and D. B. Jones. 1992. á1 antitrypsin gene exon use in stimulated lymphocytes. J Clin Pathol 45:776-780.
17. Winkler, I. G., J. Hendy, P. Coughlin, A. Horvath, and J.-P. Levesque. 2005. Serine protease inhhibitors serpinal and serpina3 are down-regulated in bone marrow during hematopoietic progenitor mobilization. J. Exp. Med. 201:1077-1088.
18. Bayer,HealthCare. Prolastin Product Monograph.www.talecris.com. 2003.
19. Bristow, C. L., F. di Meo, and R. R. Arnold. 1998 Specific activity of á1 proteinase inhibitor and á2macroglobulin in human serum: Application to insulin-dependent diabetes mellitus. Clin. Immunol. Immunopathol. 89:247-259.
20. Krutzik, P. O. and G. P. Nolan 2006. Fluorescent cell barcoding in flow cytometry allows high-throughput drug screening and signaling profiling. Nat Meth 3:361-368.
Bristow, C. L., Cortes, J., Mukbtarzad, R. Trucy, M., Franklin, A., Romberg, V., and Winston, R. (2009), a1Antitrypsin therapy increases CD4+ lymphocytes to normal values in HIV-1 patients. In Soluble factors mediating innate immune responses to HIV infection, M. Alfano, ed. Bentham Science Publishers).
Bristow, C. L., di Meo, F. and Arnold, R. R. (1998). Specific activity of α1proteinase inhibitor and α2macroglobulin in human serum. Application to insulin-dependent diabetes mellitus. Clin. Immunol. Immunopathol. 89, 247-259.
Bristow, C. L., Fiscus, S. A., Flood, P. M., and Arnold, R. R. (1995). inhibition of HIV-1 by modification of a host membrane protease. Int. Immunol. 7, 239-249.
Bristow, C. L., Mercatante, D. R., and Kole, R, (2003). HIV-1 preferentially binds receptors co-patched with cell surface elastase. Blood 102. 4479-4486.
Bristow, C. L., Patel, H., and Arnold, R. R. (2001). Self antigen prognostic for human immunodeficiency virus disease progression. Clin Diagn. Lab. Immunol. 8, 937-942.
Cao, C., Lawrence, D. A., Li, Y., Von Amin, C. A., Herz, J., Su, E. J., Makarova, A., Hyman, B. T., Strickland, D. K., and Zhang, L. (2006). Endocytic receptor LRP together with tPA and PAI-1 coordinates Mac-1-dependent macrophage migration. EMBO J. 25, 1860-1870.
Castro, K. G., Ward, J. W., Slutsker, L., Buehler, J. W., Jaffe, J., Berkelman, R. L., and Curran, J. W. (1992). 1993 revised classification system for HIV infection and expanded surveillance case definition for AIDS among adolescents and adults. Morbid. Mortal. Weekly Rep. 41, 1-19.
Cselenyi, C. S., Jernigan, K. K., Tahinci, E., Thorn, C. A., Lee, L. A., and Lee, E. (2008). LRP6 transduces a canonical Wnt signal independently of Axin degradation by inhibiting GSK3's phosphorylation of +1-catenin. PNAS. 105, 8032-8037.
Dimitrov, S., Benedict, C., Heutling, D., Westermann, J., Born, J., and Lange, T. (2009). Cortisol and epinephrine control opposing circadian rhythms in T-cell subsets. Blood-2008.
Donskoy, E., Foss, D., and Goldschneider, I. (2003). Gated importation of prothymocytes by adult mouse thymus is coordinated with their periodic mobilization from bone marrow. J. Immunol. 171, 3568-3575.
Goselink, H. M., van Damme, J., Hiemstra, P. S., Wuyts, A., Stolk, J., Fibbe, W. E., Willemze, R., and Falkenburg, J. H. (1996). Colony growth of human hematopoietic progenitor cells in the absence of serum is supported by a proteinase inhibitor identified as antileukoproteinase. J. Exp. Med. 184, 1305-1312.
Huang, J.-H., Qi, Z., Wu, F., Kotula, L., Jiang, S., and Chen, Y.-H. (2008). Interaction of HIV-1 gp41 Core with NPF Motif in Epsin: Implication in endocytosis of HIV. J. Biol. Chem. 283, 14994-15002.
Hubner, W., McNerney, G., Chen, P., Dale, B. M., Gordon, R. E., Chuang, F. Y. S., Li, S.-D., Asmuth, D. M., Huser, T., and Chen, B. K. (2009). Quantitative 3D Video Microscopy of HIV Transfer Across T Cell Virological Synapses. Science 323, 1743-1747.
Lapidot, T. and Petit, I. (2002). Current understanding of stem cell mobilization: The roles of chemokines, proteolytic enzymes, adhesion molecules, cytokines, and stromal cells. Exp. Hematol. 30, 973-981.
Munch, J., Standker, L., Adermann, K., Schulz, A., Schindler, M., Chinnadurai, R., Pohlmann, S., Chaipan, C., Biet, T., Peters, T., Meyer, B., Wilhelm, D., Lu, H., Jing, W., Jiang, S., Forssmann, W. G., and Kirchhoff, F. (2007). Discovery and Optimization of a Natural HIV-1 Entry Inhibitor Targeting the gp41 Fusion Peptide. Cell 129, 263-275.
Rusche, J. R., Javaherian, K., McDanal, C., Petro, J., Lynn, D. L., Grimaila, R., Langlois, A., Gall, R. C., Arthur, L. O., Fischinger, P. J., Bolognesi, D. P., Putney, S. D., and Matthews, T. J. (1988). Antibodies that inhibit fusion of human immunodeficiency virus-infected cells bind a 24 amino acid sequence of the viral envelope gp120. Proc Natl Acad Sci USA 85, 3198-3202.
Shapiro, L., Pott, G. B., and Ralston, A. H. (2001). Alpha-1-antitrypsin inhibits human immunodeficiency virus type 1. FASEB J. 15, 115-122.
Shulman, Z., Shinder, V., Klein, E., Grabovsky, V., Yeger, O., Geron, E., Montresor, A., Bolomini-Vittori, M., Feigelson, S. W., Kirchhausen, T., Laudanna, C., Shakhar, G., and Alon, R. (2009). Lymphocyte Crawling and Transendothelial Migration Require Chemokine Triggering of High-Affinity LFA-1 Integrin. Immunity 30, 384-396.

1-74. (canceled)

75. A kit for determining the number of CD4+ T cells in a sample comprising, in one or more separate receptacles, elastase, an elastase detection agent, diluents and instructions for use.

76. The kit of claim 75 wherein said elastase is porcine pancreatic elastase (PPE).

77. The kit of claim 75 wherein said elastase detection agent is the colorimetric elastase-specific peptide substrate succinyl L-Ala-L-Ala-L-Ala-p-nitroanalide (SA3NA).

78. The kit of claim 77 further comprising alpha-1 proteinase inhibitor (α1PI) and a reagent for determining the concentration of α1PI in a sample.

79. The kit of claim 78 wherein said sample is a member selected from the group consisting of urine, blood, serum and plasma.
